(19) United States
(12) Patent Application Publication
(10) Pub. No.: US 2001/0010814 A1
(43) Pub. Date: Aug. 2, 2001

(54) ADENOVIRAL VECTOR ENCODING PRO-APOPTOTIC BAX GENE AND USES THEREOF

(76) Inventors: DAVID T. CURIEL, BIRMINGHAM, AL (US); JESUS GOMEZ-NAVARRO, BIRMINGHAM, AL (US); JIALING XIANG, BIRMINGHAM, AL (US)

Correspondence Address: BENJAMIN A. ADLER, 8011 CANDLE LANE, HOUSTON, TX, 77071, US

(21) Appl. No.: 09/393,173

(22) Filed: Sep. 9, 1999

Related U.S. Application Data
(60) Provisional application No. 60/097,732, filed on Aug. 24, 1998.
(60) Provisional application No. 60/101,868, filed on Sep. 25, 1998.

(51) Int. Cl. A61K 48/00

(52) U.S. Cl. 424/93.1; 514/44

(57) ABSTRACT

The present invention provides a pharmaceutical composition, comprising a recombinant adenoviral vector encoding an pro-apoptotic Bax gene and a pharmaceutically acceptable carrier. Further is provided a method of treating an individual having a pathophysiological state, comprising the step of administering to said individual a pharmacologically effective dose of this composition.

### CROSS-REFERENCE TO RELATED APPLICATION

This application claims benefit of priority under 35 USC §119(e) of U.S. provisional applications Ser. No. 60/097,732 filed Sep. 10, 1998 and Ser. No. 60/101,868 filed Sep. 25, 1998, now abandoned.

### FEDERAL FUNDING LEGEND

This invention was created in part using funds from the National Institutes of Health under grants RO1-CA68245-02. The federal government, therefore, has certain rights in this invention.

### BACKGROUND OF THE INVENTION

1. Field of the Invention

The present invention relates generally to the fields of gene therapy and transplantation. More specifically, the present invention relates to an adenoviral vector encoding an pro-apoptotic bax gene for gene therapy.

2. Description of the Related Art

Ovarian cancer is one of the most common malignancies among American women and still remains the most lethal of gynecological cancers. An important feature of ovarian cancer is the inactivation of normal tumor supressor genes. The p53 tumor suppressor gene is commonly altered in ovarian cancer (1). Loss of p53 is associated with the resistance of tumor cells to chemotheraputic agents and with a worse prognosis (1). Restoration of wild-type p53 gene via recombinant adenovirus can prolong survival in a murine xenograft model of human ovarian carcinoma (2), probably due to its apoptotic effects. It is known that p53-mediated cell death is due, at least in part, to through upregulation of bax, a pro-apoptotic member of the Bcl-2 family. This suggests that bax may be a component of p53-mediated apoptosis (3).

Recently, two novel biological features of bax have been described. First, bax can induce apoptosis in a Bcl-2-independent manner (4), probably through formation of specific ion channels on mitochondria membranes, leading to mitochondrial dysfunction (5, 6). Second, bax can function as a tumor suppressor (7). Overexpression of bax suppresses tumorigenesis in a transgenic mouse model system (7). Bax-deficient mice display hyperplasia of neurons, lymphocytes, and ovarian granulosa cells (8).

Downregulation of bax expression is thought to confer a growth advantage by rendering cells resistant to chemotherapy-induced apoptosis. Furthermore, mutations of bax have been found in many types of tumors, including breast, colon and ovarian cancers, and in haemopoietic malignancies (9, 10).

The prior art is deficient in the lack of effective means of gene therapy using an adenoviral vector encoding an pro-apoptotic bax gene. The present invention fulfills this longstanding need and desire in the art.

### SUMMARY OF THE INVENTION

Bax is a pro-apoptotic member of the Bcl-2 family which can act as a tumor suppressor. In this study, an inducible recombinant adenoviral vector encoding bax, was generated employing inducibility via the Cre-loxP system. Bax expression was specifically induced by Cre recombinase and resulted in apoptotic cell death in human ovarian cancer cells. In contrast, expression of bax did not induce cell death in normal human peritoneal mesothelial cells. These results indicate that bax may preferentially induce cell death in human ovarian cancer cells, suggesting that this approach might be of utility in gene therapy for ovarian cancer.

In one embodiment of the present invention, there is provided a recombinant adenoviral vector encoding an pro-apoptotic bax gene.

In another embodiment of the present invention, there is provided a method of treating an individual having a pathophysiological state, comprising the step of administering to said individual a pharmacologically effective dose of the composition of the present invention.

In another embodiment of the present invention, there is provided a pharmaceutical composition, comprising the adenoviral vector encoding an pro-apoptotic bax gene and a pharmaceutically acceptable carrier.

In yet another embodiment of the present invention, there is provided a method of treating an individual having ovarian cancer, comprising the step of administering to said individual a pharmacologically effective dose of a pharmaceutical composition, comprising a recombinant adenoviral vector encoding an pro-apoptotic Bax gene and a pharmaceutically acceptable carrier.

In yet another embodiment of the present invention, there is provided a method of sensitizing tumor cells to chemotherapy and/or radiotherapy in an individual, comprising the step of administering to said individual a pharmacologically effective dose of a pharmaceutical composition, comprising a recombinant adenoviral vector encoding an pro-apoptotic bax gene and a pharmaceutically acceptable carrier.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1A shows schema of the inducible recombinant Ad/bax construct. HA-tagged bax cDNA was inserted in the SwaI site of pΔEloxP vector (13). In the presence of Cre recombinase, excision of the loxP-neo^{r}-loxP cassette allows appropriate transcription/translation of the bax gene. FIG. 1A and FIG. 1B shows an immunoblot analysis of bax expression in human ovarian cancer cell lines. OVCAR3 (FIG. 1A) and SKOV3ip1 (FIG. 1B) cells were infected at an MOI of 100 with Ad/GFP (lane 1); Ad/bax (lane 2); Ad/bax plus Ad/Cre (lane 3); and Ad/GFP plus Ad/Cre (lane 4). The proteins (10 μg per lane) were separated on a 12% SDS-PAGE gel, followed by immunoblotting with an anti-HA antibody.

FIG. 2 shows the cell death induced by bax expression. OVCAR3 cells were infected with Ad/bax plus Ad/Cre, Ad/bax plus Ad/GFP, or left uninfected (no virus). Aliquots of cells were taken out at various times as indicated and stained with trypan blue to analyze for cell viability (FIG. 2A). A separated aliquot of cells was subjected to immunoblot analysis and probed with either an anti-HA mAb or an anti-Bcl-2 antibody (FIG. 2B). This is a representative experiment of two independent experiments.

FIG. 3 shows Ad/bax -mediated apoptosis in human ovarian cancer cells. OVCAR3 cells were infected with Ad/bax or Ad/bax plus Ad/Cre as indicated. In FIG. 3A, samples of cells were stained with H33258 and visualized by UV light microscopy. In FIG. 3B, cells were lysed and 20 μg proteins from each sample were subjected to measure the caspase 3 activity using the fluorogenic substrate, DEVD-AFC. The caspase activity is shown by Relative Fluorescent Unit (RFU).

FIG. 4 shows Ad/bax preferentially induces death in the human ovarian cancer cells. Normal human mesothelial cells (NPL-CR3 and 4) and human ovarian cancer cell lines (OVCAR3 and SKOV3ip1) were plated in 12 well plates and infected with the recombinant adenovirus as indicated (MOI: 100 for OVCAR3, and 300 SKOV3ip1 and the mesothelial cells). Forty hours post-infection, the cells were stained with crystal violet to visualize cell viability (FIG. 4A). To assay cell survival, the cells were harvested and scored by trypan blue exclusion (FIG. 4B). The results show the mean of experiments performed in duplicate.

FIG. 5 shows the construction and expression of the inducible recombinant Ad/Bax vector. FIG. 5A shows a schema of the inducible recombinant Ad/Bax construct. Ha-tagged bax cDNA was inserted in the SwaI site of pΔEloxP vector. In the presence of Cre recombinase, excision of the loxP-neor-loxP cassette allows appropriate transcription/translation of the bax gene. FIG. 5B shows an immunoblot analysis of Bax protein expression in human ovarian cancer cell lines. SKOV3.ip1 and SW626 cells were infected at an moi of 100 with Ad/GFP (lane 1); Ad/Bax (lane 2); Ad/Bax plus Ad/Cre (lane 3); and Ad/GFP plus Ad/Cre (lane 4). The proteins (10 μg per lane) were separated on a 12% SDS-PAGE gel, followed by immunoblot with an anti-HA antibody.

FIG. 6 shows adenovirus-mediated expression of Bax radiosensitizes an ovarian cancer cell line. FIG. 6A: cell viability after combined treatment. SKOV3.ip1, SW626 and primary ascites-derived cells were infected with Ad/Bax, Ad/Cre, or both, or left uninfected (mock). Then, cells were irradiated with a dose ranging form 0 to 8 Gy. Cell viability was evaluated 3 (primary cells), 5 (SKOV3.ip1) or 8 (SW626) days later by staining with crystal violet. FIG. 6B: dose and time dependency of cell death. SKOV3.ip1 cells were infected with Ad/Bax and Adluc, or Ad/Bax and Ad/Cre, or left uninfected (mock). Then, cells were irradiated with a dose of 0, 4, or 8 Gy. Cell death was evaluated 24, 48, and 72 hr later by FACS analysis. Cells positively stained with propidium iodide or annexin V were counted. Results of one experiment, repeated twice with similar findings, are displayed as the mean ± standard deviation of three samples per group. FIG. 6C: radiosensitization as determined by a standard colony-forming assay. SKOV3.ip1 cells were infected with Ad/Bax, Ad/Cre, or both, or left uninfected (mock). Then, cells were irradiated with a dose ranging form 0 to 8 Gy. After a similar treatment that included different viral doses, cells were plated for colony formation, and stained two weeks later for cell viability with crystal violet. Colonies containing more than 50 cells were counted as viable. Representative results of one experiment repeated three times with similar results are depicted. FIG. 6D: radiosensitization of SW626 cell line as determined by proliferation assay. Cells were infected with Ad/Bax and Ad/Cre or irrelevant virus 24 hrs after radiation with 0 to 8 Gy, and then were counted at different time points.

FIG. 7 shows the radiosensitization by Ad/Bax in a human ovarian cancer cell line involves phenotypical changes typical of apoptosis. SKOV3.ip1 cells were infected with Ad/Bax plus Ad/Cre or mock infected as indicated. Later, cells were treated with 8 Gy of radiation. Samples of cells were stained with H33258 and visualized by UV light microscopy.

FIG. 8 shows the mechanisms of cell death in a human ovarian cancer cell line after combined treatment with radiation and Ad/Bax. SKOV3.ip1 cells were infected with Ad/Bax plus Ad/Cre or mock infected as indicated. Later, cells were treated with a radiation dose ranging from 0 to 8 Gy of radiation. At 24 hr post-radiation, samples of cells were stained with Annexin-V to detect externalized phosphatidylserine and counted by fluorescence-activated cell sorter analysis. Propidium iodide was added to allow also determination of cells undergoing necrosis. Differences between relevant groups, including treatment with Ad/Bax and Ad/Cre without radiation versus same treatment with 8 Gy radiation, and treatment with 8 Gy radiation with and without virus, were both statistically significant (p<0.00001), and are highlighted (* and **). Similar statistically significant differences were observed at 72 hr, with higher absolute levels of cell death.

FIG. 9 shows the tumor nodule formation after cell treatment with Bax and radiation. SKOV3.ip1 cells were infected with Ad/Bax plus Ad/Cre, control Ad/CD virus, or mock infected as indicated. Later, cells were treated with a radiation dose ranging from 0 to 8 Gy of radiation. At 24 hr post-radiation, cells were injected subcutaneously into both upper flanks with 20×10⁶ SKOV3ip1 cells, and then followed for nodule formation every other day. Quantitative data obtained at 6 weeks is presented in a chart FIG. 9A: (◯, radiation alone; ▴, Ad/CD alone; ●, Ad/CD with radiation; ♦, Ad/Bax+Ad/Cre alone; [symbol] , Ad/Bax+Ad/Cre with radiation). A photograph of representative animals obtained at 8 weeks post-injection is also presented (FIG. 9B).

FIG. 10A shows schema of the inducible recombinant adenoviral vector encoding bax. FIG. 10B shows a Western blot analysis of expression of Bax by inducible adenoviral system in ovarian cancer cell lines. A panel of ovarian cancer cell lines (SKOV3.ip1, OV4, OVCAR3, SW626, and PA-1) were infected with Ad/Bax plus Ad/Cre, or Ad/LacZ plus Ad/Cre (ratio 5 to 1 at a total moi of 100). After a 24 hour-infection, the cells were collected and lysed in NP-40 lysis buffer. Thirty micrograms of protein from the lysates were subjected to 12% SDS-gel electrophoresis and transferred to a PVDF membrane. The expression of hemagglutinin (HA) tagged-Bax protein was revealed by an anti-HA monoclonal antibody and visualized by enhanced chemoluminescence. Molecular weight markers are indicated at the left in kilodalton.

FIG. 11 shows Bax-mediated cell death in ovarian cancer cell lines. A panel of ovarian cancer cell lines (SKOV3.ip1, OV4, OVCAR3, SW626, and PA-1) were infected with or without the following viruses: (1) no virus, (2) Ad/Bax plus Ad/Cre, and (3) Ad/LacZ plus Ad/Cre, as described in FIG. 10B. After a 48 hours-infection, the cell viability was measured by an MTS assay. Non-virus treated cells were used as 100% viable control for this experiment.

FIG. 12 shows examination of endogenous Bax and Bcl-2 protein levels and status of p53. Fifty micrograms of protein lysates from each of the ovarian cell lines SKOV3.ip1, OV4, OVCAR3, SW626, and PA-1 were subjected to immunoblotting analysis with an anti-Bax polyclonal antibody, an anti-Bcl-2 monoclonal antibody, or an anti-p53 monoclonal antibody. The p53 status was determined.

FIG. 13 shows Bax-mediated apoptosis in primary ovarian cancer cells. The primary ovarian cancer cells were purified from the ascites of ovarian cancer patients as described below. After recovery, the cells were infected with Ad/Bax plus Ad/Cre or Ad/LacZ plus Ad/Cre (at a ratio of 5 to 1 and a total moi of 500) for 24 to 48 hours. The expression of Bax was detected by immunoblotting with an anti-HA monoclonal antibody. Molecular weight markers are indicated at the left in kilodalton (FIG. 13A). The apoptotic cells were visualized by nuclear staining with H33258 under the UV microscope (FIG. 13B).

FIG. 14 shows enhancement of the cytotoxic effect of taxol by adenovirus-mediated transfer of the bax gene into ovarian cancer cell lines. Ovarian cancer cell lines, SKOV3.ip1 and SW626 were infected with Ad/Bax plus Ad/Cre or Ad/LacZ plus Ad/Cre at a ratio of 5 to 1 and with a total moi of 100, or left without viral infection. One hour after viral infection, the cells were washed and treated with taxol at various concentrations, as indicated, for additional 48 hours. The cell viability was measured by an MTS assay.

FIG. 15 enhancement of the cytotoxic effect of taxol by Bax in ovarian cancer primary cells. The primary ovarian cancer cells were isolated as described in the Methods. Approximately 8×10³ cells were plated in each well of a 96-well plate one day before the start of the experiments. (FIG. 15A), For the single treatment of Bax, the cells were infected with Ad/Bax plus Ad/Cre or Ad/LacZ plus Ad/Cre at a ratio of 5 to 1 with different doses of viruses as indicated. (FIG. 15B): For combined treatment, the cells were treated under the same condition as in (FIG. 15A) but with a fixed total moi of 200 for 2 hours. After infection, the cells were washed and incubated with or without taxol for additional 48 hours. The viability of the cells from both experiments was measured by an MTS assay.

### DETAILED DESCRIPTION OF THE INVENTION

In an effort to investigate whether bax can function as a tumor suppressor in human ovarian cancer, the bax gene was overexpressed in a recombinant adenoviral vector using the Cre-loxP inducible system. Expression of bax preferentially induced apoptosis in human ovarian cancer cells, but not in normal peritoneal mesothelial cells which line the body cavity in which the ovary resides. These results suggest that the recombinant bax adenovirus would be of utility in gene therapy for ovarian cancer.

The following abbreviations may be used herein: EGF, epidermal growth factor; Ad/Bax, recombinant bax adenovirus; GFP, green fluorescence protein; PCR, polymerase chain reaction; MOI, multiplicity of infection; DEVD-AFC, carbobenzoxy-Asp-Glu-Val-Asp-7-amino-4-trifluoromethyl coumarin; HRP, horseradish peroxidase.

The present invention is directed to a recombinant adenoviral vector encoding an pro-apoptotic bax gene. Further, the present invention is directed to a pharmaceutical composition, comprising the adenoviral vector described herein and a pharmaceutically acceptable carrier.

The present invention is also directed to a method of treating an individual having a pathophysiological state, comprising the step of administering to said individual a pharmacologically effective dose of the composition described herein. In one aspect, the pathophysiological state is a neoplastic disease. Representative neoplastic diseases include breast cancer, colon cancer, ovarian cancer, glioma, osteosarcoma and haemopoietic cancers. To treat these diseases, the composition is preferably administered in a dose of from about 0.1 mg/kg to about 100 mg/kg.

The present invention is also directed to a method of treating an individual having ovarian cancer, comprising the step of administering to said individual a pharmacologically effective dose of a pharmaceutical composition, comprising a recombinant adenoviral vector encoding an pro-apoptotic bax gene and a pharmaceutically acceptable carrier. Preferably, the composition is administered in a dose of from about 0.1 mg/kg to about 100 mg/kg.

The following examples are given for the purpose of illustrating various embodiments of the invention and are not meant to limit the present invention in any fashion.

### EXAMPLE 1

### Cell Culture

The human embryonic kidney cell line 293 and the human ovarian cancer cell line NIH:OVCAR3 were obtained from the American Type Culture Collection (ATCC, Rockville, Md.). The human ovarian cancer cell line SKOV3ip1 was from Janet Price (M.D. Anderson Cancer Center, Houston, Tex.). SKOV3ip1 and 293 cells were grown in DMEM/Ham's F12 medium supplemented with 10% FCS, 100 U/ml of penicillin, and 100 μg/ml streptomycin. OVCAR3 cells were maintained in RPMI 1640 medium with 20% FCS, and 10 μg/ml insulin. The normal human mesothelial cells were isolated from the fresh peritoneum lining tissue of healthy patients undergoing surgery for unrelated reasons by mechanical disruption and collagenase D treatment (Boehringer Mannheim, Indianapolis, Ind.) and were maintained in DMEM/Ham's F12 medium supplemented with 10 ng/ml EGF⁴ (Gibco BRL, Gaithersburgh, Md.) and 0.4 μg/ml hydrocortisone (Sigma, St Louis, Mo.) (11). The purity of the mesothelial cells was determined by immunophenotyping the cell population and demonstrating immunoreactivity with antibodies directed against broad spectrum cytokeratins, calretinin, and nonreactivity for fibroblast, lymphoid, and histiocytic markers.

### EXAMPLE 2

### Construction and Inducible Expression of Bax with Recombinant Adenoviruses

To construct an inducible recombinant adenoviral vector containing the bax cDNA, the Cre-loxP system was used (12). The hemagglutinin (HA)-tagged full length bax cDNA (5) was cloned into the SwaI site of pØEloxP (13) to generate the bax/pΔEloxP shuttle plasmid. The bax/pΔEloxP and pJM17 (Microbix, Inc., Ontario, Canada) plasmids were cotransfected into 293 cell using the Superfect lipid-based transfection method (Qiagen, Chatsworth, Calif.) according to the manufacturer's protocol. Plaques were screened for recombinant bax adenoviral vector (Ad/Bax) using PCR. The recombinant Ad/bax was isolated and purified from a single plaque as described previously (13). Viral titer was measured by standard plaque assay using 293 cells. For induction of bax proteins, cells were coinfected with Ad/bax and Ad/Cre, a recombinant adenoviral vector encoding Cre recombinase (14). An MOI of 100 or 300 was used for infection. An equal amount of Ad/GFP was used as a transduction control for all experiments.

### EXAMPLE 3

### Apoptosis Assay

Apoptosis was measured by cell viability, nuclear morphology and caspase activity. Cell viability was measured by trypan blue exclusion and crystal violet staining as previous described (3, 15). For visualization of nuclear morphology, the cells (0.5×10⁶) were stained with the intercalating DNA dye H33258 (Sigma, St. Louis, Mo.) and observed under a UV light microscope. The catalytic activity of the caspase 3 was measured as described previously (5). The release of AFC from the fluorogenic substrate DEVD-AFC (Bio-Rad, Hercules, Calif.) was monitored with an excitation 360 nm and emission 505 nm. The optical density was read on a VersaFluor Fluorometer (Bio-Rad).

### EXAMPLE 4

### Immunoblot Analysis

Cells were lysed with an NP-40 buffer containing 0.3% NP-40, 142 mM KCl, 5 mM MgCl₂, 2 mM EDTA, 20 mM HEPES pH 7.4, and a cocktail of protease inhibitors (leupeptin 10 μg/ml, aprotinin 10 μg/ml, and 2 mM EGTA). Total proteins (10-50 μg) were separated on a 12% SDS-polyacrylamide gel and subsequently transferred to a PVDF membrane (Bio-Rad). The membranes were blocked with 2% non-fat dry milk and probed with a bax polyclonal antibody at a dilution of 1:2000 (Santa Cruz Biotechnology, Santa Cruz, Calif.), an HA monoclonal antibody at a dilution of 1:1000 (Santa Cruz), or a Bcl-2 antibody at a dilution of 1:2000 (DAKO, Carpinteria, Calif.). After washing with TBS-Tween (50 mM Tris-HCl, pH 8.3, 150 mM NaCl, and 0.5% Tween-20), an HRP conjugated secondary antibody was then added, followed by detection with an enhanced chemiluminescence system (Renaissance; NEN, Boston, Mass.).

### EXAMPLE 5

### Generation of an Inducible Recombinant Adenoviral Vector Encoding Bax using the Cre-loxP System

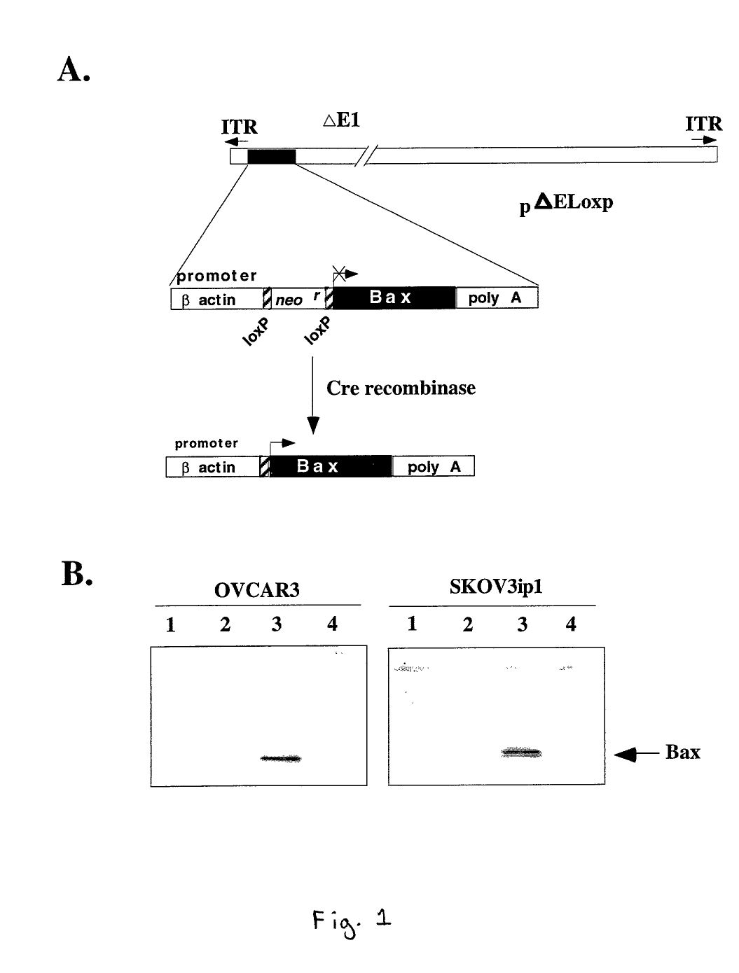

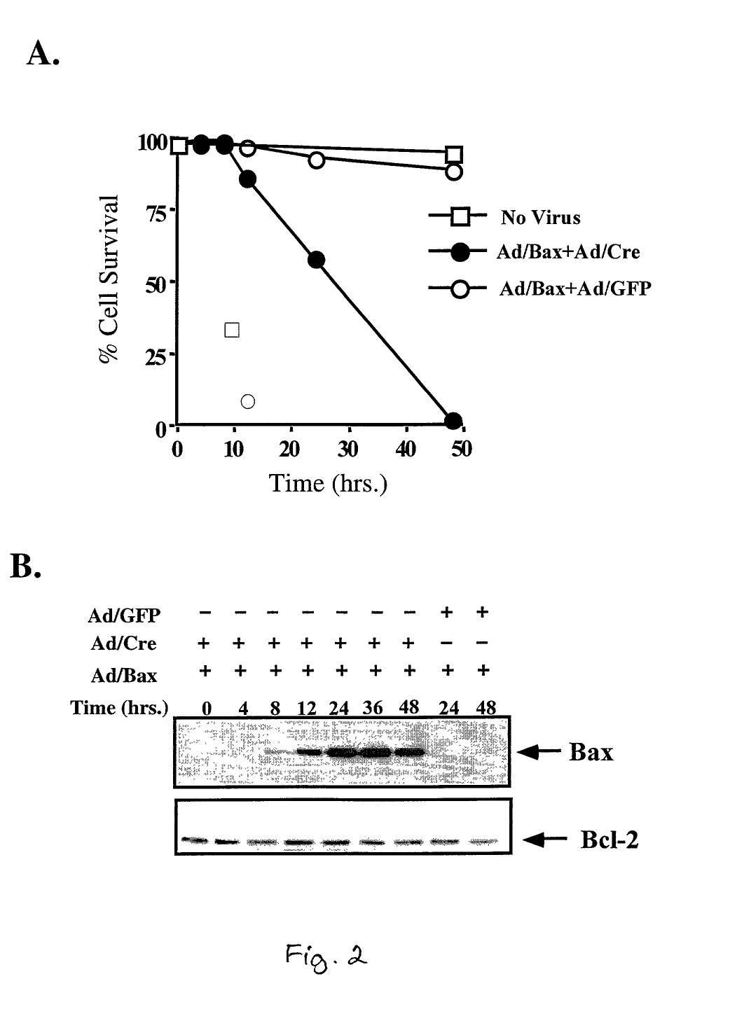

Overexpression of bax is known to be cytotoxic in many cell types (5). It is difficult, therefore, to generate a stable cell line expressing high levels of bax. To overcome this problem, the Cre-loxP system was employed to generate an inducible recombinant bax adenoviral vector (Ad/Bax). The HA-tagged bax coding sequence was placed under the control of the β-actin promoter and a downstream loxP-neo^{r}-loxP excision cassette in pΔEloxP shuttle vector (13). The loxP-neo^{r}-loxP cassette is composed of a neo^{r} gene flanked by two head-to-tail loxP sites which disrupt the promoter/coding-region structure required for bax expression. In this system, the bax gene can not be translated until the loxP-neo^{r}-loxP cassette is excised by Cre recombinase (FIG. 1A). To produce the recombinant adenovirus, pΔEloxP/bax and pJM17 were cotransfected into 293 cells and several plaques were screened by PCR. Expression of bax proteins was confirmed by coinfection of cells with Ad/bax plus Ad/Cre followed by immunoblotting analysis with an anti-HA monoclonal antibody.

To test the inducibility and stringency of the Cre-loxP system, two human ovarian cancer cell lines, OVCAR3 and SKOV3ip1, were infected with Ad/bax with or without Ad/Cre. The cells were harvested at 24 or 48 hours post-infection. Immunoblotting analysis revealed that expression of the HA-bax protein could be detected only in cells coinfected with Ad/bax and Ad/Cre, but not in cells infected with Ad/bax alone or with a control adenoviruses expressing GFP (FIGS. 1B and C). The efficiency of adenoviral infection of OVCAR3 and SKOV3ip1 cells was very high; more than 95% of cells tansduced with a single dose of Ad/GFP at an MOI of 100 appeared green cells under a fluorescent microscope 2 days post-infection (data not shown). Thus, the inducible expression of bax by the Ad/bax virus appears to be specific with minimal leakage in the absence of Cre recombinase.

### EXAMPLE 6

### Induction of Bax Expression Causes Cell Death

Bax has been shown to function as a pro-apoptotic molecule by counteracting Bcl-2 or by directly killing cells (3, 4). To determine the biological activity of recombinant Ad/bax, the human ovarian cancer cell lines, OVCAR3 and SKOV3ip1, were coinfected with Ad/bax or Ad/GFP plus Ad/Cre for various periods of time. Coinfection of OVCAR3 cells with Ad/bax plus Ad/Cre caused massive cell death as shown by trypan blue exclusion assays (FIG. 2A). Cell death could be detected as early as 12 hours after infection and nearly 90% of the cells died within 48 hours (FIG. 2A). The time course of cell death correlated well with the time course of Ad/bax expression, which was detected as early as 8 hours post-infection, and peaked at 24 to 48 hours (FIG. 2B). On the other hand, the level of Bcl-2 remained unchanged during this time (FIG. 2B). Similar results were obtained with SKOV3ip1 cells. This demonstrates that Ad/bax induces cell death in human ovarian cancer cells and this killing is not due to dysregulation of Bcl-2 expression.

### EXAMPLE 7

### Ad/bax Induces Apoptosis in Ovarian Cancer Cells

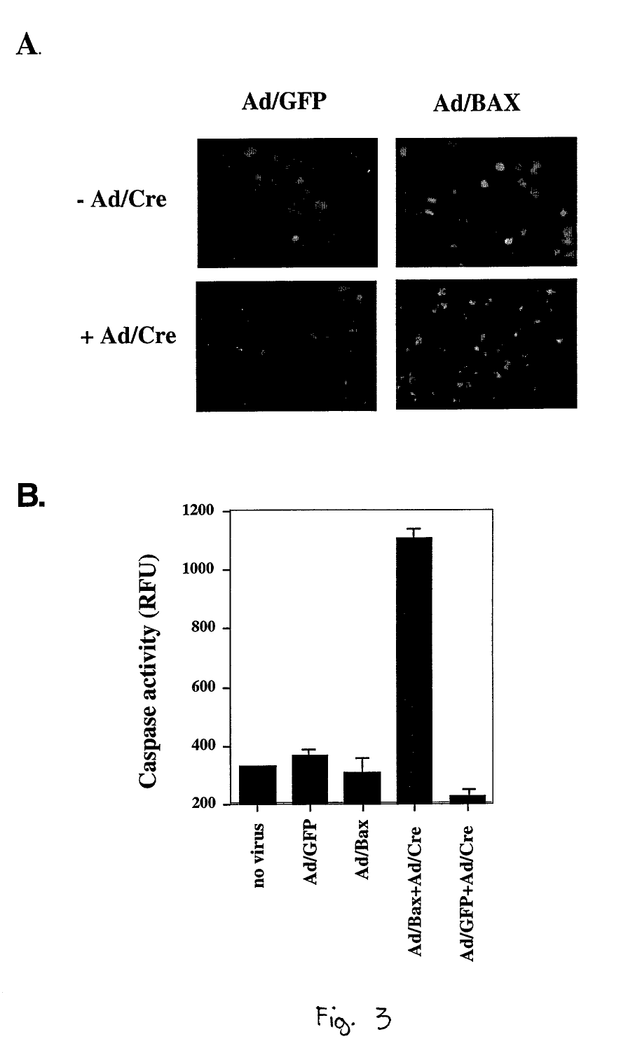

To determine whether Ad/bax-mediated cell death occurs through apoptosis, OVCAR3 cells were coinfected with Ad/bax or Ad/GFP plus Ad/Cre for various periods of time. Cell morphology was examined by brightfield microscopy. Cells coinfected with Ad/bax and Ad/Cre became rounded, shrank, subsequently detached from the tissue culture plates, and died. Nuclear staining with the intercalating DNA dye H33258 revealed that up to 90% of cells displayed nuclear condensation and fragmentation 48 hours post-infection, as analyzed by a UV microscopy (FIG. 3A). Furthermore, the activity of caspase 3 was significantly increased in cells coinfected with Ad/bax and Ad/Cre, as measured by a fluorogenic caspase 3 substrate, DEVD-AFC (FIG. 3B). Similar results were obtained with SKOV3ip1 cells. These results thus indicate that Ad/bax induces apoptotic cell death in human ovarian cancer cells and utilizes the same proteolytic apoptotic pathway as non-viral bax expression system.

### EXAMPLE 8

### Ad/bax Preferentially Induces Apoptosis in Ovarian Cancer Cells

Epithelial ovarian cancer is the leading cause of death from malignant gynecologic tumors. At the time of diagnosis approximately 75% of patients have advanced intraperitoneal metastasis (16). Metastatic implants are attached to the peritoneal lining by interaction of the CD44 receptor of ovarian cancer cells to hyalurouic acid present in the matrix at the surface of mesothelial cells (17).

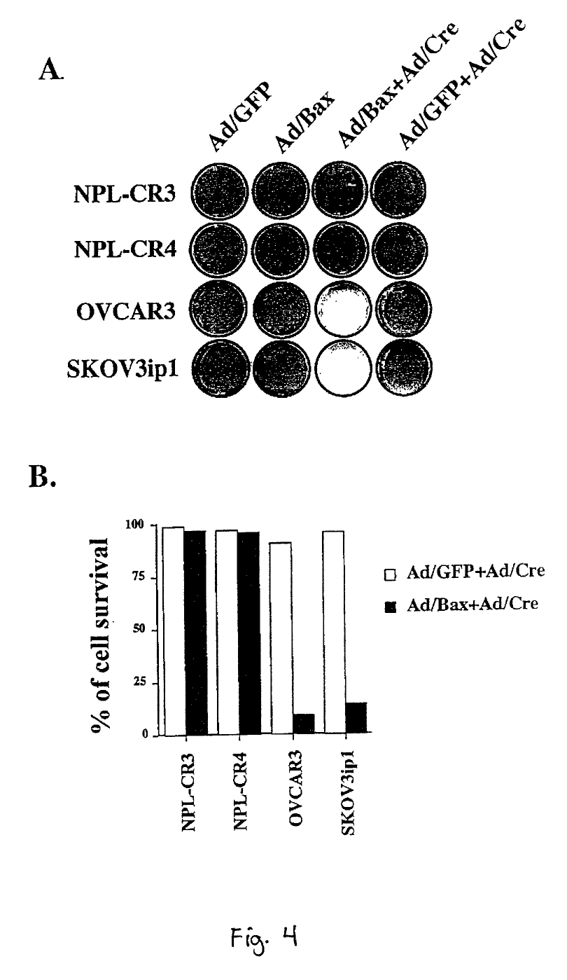

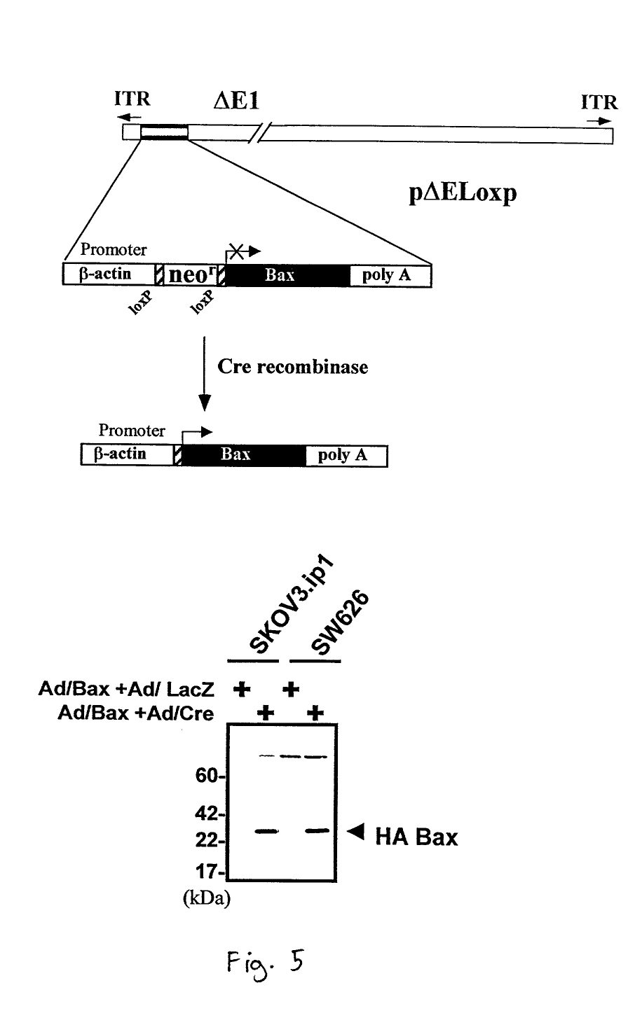

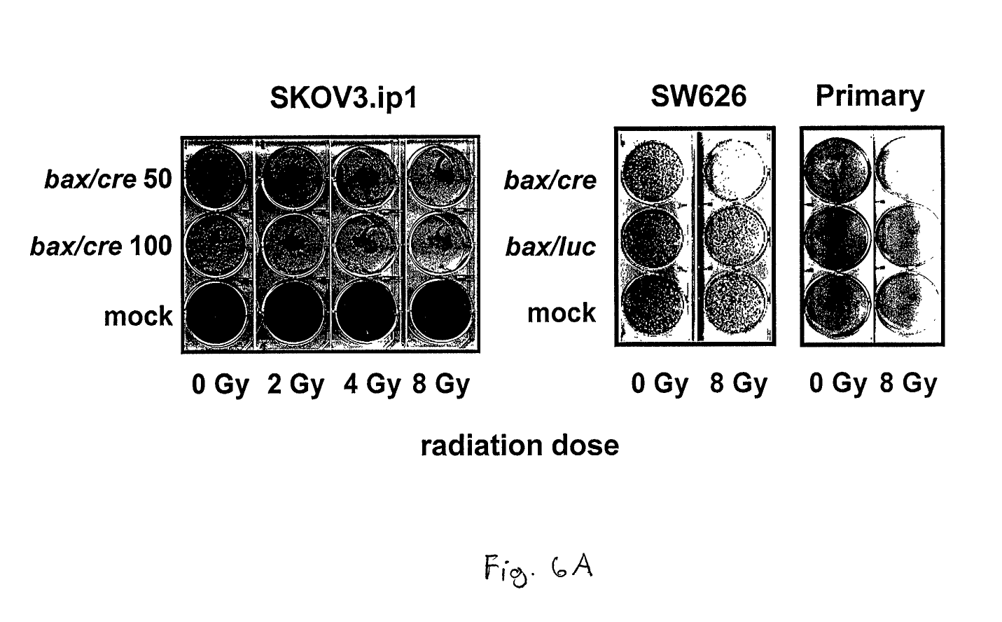

To evaluate the specificity of Ad/bax-mediated apoptosis, normal peritoneal mesothelial cells (NPL-CR3 and NPL-CR4), and ovarian cancer cells (OVCAR3 and SKOV3ip1) were coinfected with Ad/bax or Ad/GFP plus Ad/Cre, as indicated. Cell death was measured by crystal violet staining 48 hours post-infection. Massive cell death was detected in the ovarian cancer cells but not in mesothelial cells (FIGS. 4A and B).

To determine whether the mesothelial cells were susceptible to adenoviral infection, the cells were infected with Ad/GFP. Expression of GFP was determined using a fluorescent microscope. Over 90% of the ovarian cancer cells and approximately 50% of mesothelial cells were expressed GFP 48 hours post-infection with Ad/GFP, suggesting that these mesothelial cells were slightly less susceptible than the ovarian cancer cells to adenoviral infection. Nonetheless, no cell death could be detected in the mesothelial cells after bax induction. It appears that bax may be a specifically cytotoxic to the cancer cells. Taken together, the fact that Bax induces cell death in ovarian cancer cells, and the resistance of normal human mesothelial cells to bax-mediated apoptosis, suggest the potential utility of the recombinant Ad/bax in gene therapy for ovarian cancer.

### EXAMPLE 9

### Use of an Adenoviral Vector Encoding Pro-apoptotic Bax Gene for Sensitization of Tumor Cells to Chemotherapy and Radiotherapy

Whether a cell becomes committed to apoptosis depends upon the balance between proteins that mediate cell death, such a s BAX, and proteins that promote cell viability, such as BCL-2 or BCL-XL. In effect, overexpression of BAX has been shown to accelerate cell death after a variety of cellular insults, including growth factor deprivation and cytotoxic chemotherapeutic agents. Conversely, overexpression of BCL-2 represses the death function of BAX. Thus, the ratio of BAX to BCL-2 appears to be a critical determinant of a cell's threshold for undergoing apoptosis.

In this regard, the cytotoxic effects of many forms of chemotherapy and ionizing radiation are mediated through a final common pathway that involves the activation of apoptosis. An intact apoptotic pathway may be necessary for chemotherapy and radiation induced cell death, and abnormalities in the ability of the cell to undergo apoptosis may lead to the development of resistance to these therapeutic maneuvers. Therefore, the utility of BAX gene delivery for sensitization of tumor cells to the cytotoxic effects of chemotherapy and radiotherapy was examined. The construction of a vector endowed with highly efficient cellular transduction allowed the development a unique model system to this end.

To show the potential of Ad/BAX for radiosensitization, its effects in the radiation-refractory human ovarian cancer cell SKOV3.ip1 was studied. Using a standard colony assay, Ad/BAX achieves a two-log decrease in the number of tumor cell colonies after radiation. This effect, dramatic for a refractory cell line, was radiation-dose dependent, and was not observed in experimental groups treated with same doses of radiation and irrelevant viral vectors. Additional cell viability assays confirmed the radiosensitization effects of AdBAX. Thus, Ad/BAX has a proven potential for reverting radioresistance in human tumor cells. In addition to its use for radiosensitization, a similar strategy for the reversion of resistance of human tumors to chemotherapeutic drugs can be used by a person having ordinary skill in this art.

The use of Ad/BAX for reverting resistance to radiotherapy and chemotherapy has general applicability in the treatment of patients with malignant neoplasms. In particular, most patients with loco-regional and metastatic disease receive radiotherapy and chemotherapy as critical components of their treatment regimens. Importantly, a majority of patients with advanced disease become ultimately refractory to the cytotoxic effects of both interventions. This treatment failure eventually leads to uncontrolled tumor growth, further tumor dissemination, and death. Therefore, the use of maneuvers to revert the resistance to cytotoxic treatment has extraordinary potential, and an adenoviral vector encoding BAX has clearly shown its capacity for accomplishing this therapeutic goal. The increasing use of adenoviral vectors in clinical trials, particularly in cancer patients, will facilitate the translation of the proposed intervention into a variety of human gene therapy clinical trials.

Aberrant expression of normal cellular oncogenes or loss of function of tumor suppressors can lead to the development of human cancer. Therefore, one of the new strategies in cancer therapy is to reestablish the function of tumor suppressors by novel gene delivery methods. Bax is a pro-apoptotic member of the Bcl-2 family and can function as a tumor suppressor through induction of apoptosis. The present invention demonstrates the generation of an inducible recombinant bax adenovirus with the Cre-loxP system. Induction of bax expression preferentially induced apoptosis in human ovarian cancer cells but not in normal human mesothelial cells.

Initial attempts to generate a recombinant bax adenovirus using a non-inducible expression system were unsuccessful. This limitation is most probably due to the death of 293 cells induced by bax expression during the initial transfection. This is consistent with previous findings that bax possesses cytotoxic effects in non-viral transfection systems. The advantage of the Cre-loxP inducible expression system is that cytotoxic proteins will not be expressed until induced by the Cre recombinase. Using this inducible system, a recombinant bax adenovirus with a high viral titer and a high level of bax expression was generated.

It has been reported that a recombinant adenovirus expressing Bcl-xs, another pro-apoptotic member of the Bcl-2 family, can specifically induce cell death in several solid tumor cells but not normal bone marrow cells. The present invention shows that Ad/bax preferentially induces cell death in human ovarian cancer cells but not normal mesothelial cells.

The mechanisms by which bax and Bcl-xs differentially kill cancer cells are not known. One possibility is that bax is able to form heterodimers with Bcl-2 and neutralize the pro-apoptotic effect of Bcl-2. The more dependent the cells are on Bcl-2 for survival, the more likely they are to be sensitive to bax-induced cell death. Another possibility is that dividing cells might be more sensitive than non-dividing cells to bax induced cell death. Bax can form specific channels on the mitochondrial membrane, resulting in dysfunction of mitochondria, and initiation of apoptotic cascades. It is possible that mitochondria from actively dividing cancer cells might be more susceptible for bax targeting. Finally, the level of endogenous bax might determine the sensitivity of cells to bax-induced cell death. Mutations of bax have been found in many cancer cell lines and primary cancer cells. Lack of endogenous bax proteins due to frameshift mutation appears to be well correlated with the resistance to apoptosis in these cell lines. Restoration of wild-type bax protein was demonstrated to sensitize these cells to apoptosis. Consistent with this notion, OVCAR3 cells have no detectable endogenous bax and are very sensitive to bax-mediated cell death. A frameshift mutation of bax may be responsible for the high sensitivity of OVCAR3 cells to bax-mediated apoptosis.

Taken together, these results indicate that overexpression of bax using an inducible adenovirus system may preferentially kill ovarian cancer cells. This suggests that Ad/bax has the potential to be an important agent in gene therapy for ovarian cancer.

### EXAMPLE 10

### Cell Culture

The human ovarian carcinoma cell line SKOV3.ip1, a SKOV3 derivative cell line, was provided by Dr. Janet Price (University of Texas, M.D. Anderson Cancer Center, Houston, Tex.). This cell line was maintained in DMEM/F12 media supplemented with 10% fetal calf serum and L-glutamine (200 μg/ml), 100 U/ml of Penicillin, and 100 μg/ml streptomycin at 37° C. in a 100% humidified 5% CO2 atmosphere. The human ovarian carcinoma cell line SW626 and the human embryonic kidney cell line 293 were obtained from the American Type Culture Collection (ATCC, Rockville, Md.) and were grown in the same media used for SKOV3.ip1.

### EXAMPLE 11

### Purification of Primary Ovarian Cancer Cells

Primary ovarian cancer cells were isolated by affinity purification from ascites obtained from patients with ovarian cancer. Fresh ascites was centrifuged to collect cells in suspension. The cells were then washed and resuspended in PBS with 0.1% BSA. The CC49 antibody (provided by Dr. Jeffrey Schlom, NIH, Bethesda, Md.), which specifically recognizes the pancarcinomatous antigen TAG-72, was added to the cell suspension at a concentration of 1 μg/10⁷ cells, and incubated at 4° C. for one hour. To isolate the immuno-complex formed by the CC49 antibody and the TAG-72 expressing tumor cells, magnetic Dynabeads (Dynal Inc. Lake success, N.Y.) coated with a secondary anti-panmouse IgG antibody were added at a ratio of 4 beads/cell, according to the protocol of the manufacturer. The bead-targeted complexes were then washed and captured with a magnetic particle concentrator (Dynal, Inc.). Finally, the purified cells were released from the beads by brief trypsinization and recovered in RPMI complete medium. Pathological analysis of these purified cells showed that over 95% of the cells were ovarian carcinoma cells.

### EXAMPLE 12

### Production of Recombinant Adenoviruses

A recombinant adenoviral vector containing the bax cDNA (Ad/Bax) was generated, as previously described (8,9). For induction of Bax protein, cells were coinfected with Ad/Bax and the inducing adenovirus Ad/Cre, a recombinant adenoviral vector encoding cre recombinase. This enzyme cuts specifically at loxP sites, releases one stuffing loxP-delimited sequence, and thus reestablishes the contact between bax and its promoter, allowing active induction of expression of Bax. An equal amount of the recombinant adenovirus Adluc, from R. Gerard (University of Texas Southwestern Medical Center, Dallas, Tex.) that encodes the reporter gene luciferase, of AdGFP, encoding the reporter green fluorescent protein, or of Ad/CD, encoding cytosine deaminase, was used as transduction controls in our experiments. Viruses were propagated in 293 cells, purified by centrifugation in CsCl gradients, and plaque-titered in 293 cells following standard protocols.

### EXAMPLE 13

### Cell Survival Assays

To evaluate the cytotoxicity of radiation, of Bax overexpression, and the combination of both, one million SKOV3.ip1 cells and SW626 were plated per 6-well plate. Cells were infected with a multiplicity of infection (MOI) of 100 plaque forming units (PFU) per cell of either Ad/Bax or Ad/Cre, or a combination of 80 pfu/cells and 20 pfu/cell of each virus, respectively. Control cells were left uninfected. Immediately or after 24 hr, cells were irradiated with doses ranging from 0 to 8 Gy. Five days later, cell viability was measured both by trypan blue exclusion and by crystal violet staining.

### EXAMPLE 14

### Colony-forming Assay

To evaluate radiosensitization by Bax, two hundred thousand SKOV3.ip1 cells were plated 8 hr prior to irradiation in 6-well plates (Falcon, Franklin Lakes, N.J.). The cells were infected with 100 pfu/cell of Ad/Bax, with 100 pfu/cell of Ad/Cre, or with 80 pfu/cell of Ad/Bax and 20 pfu/cell of Ad/Cre, respectively. The cells were irradiated using a 60 Co therapy unit (Picker, Cleveland, Ohio) at a dose of 80 cGy/min 24 hr later. As an additional control, cells were infected with Ad/Bax plus Adluc. Immediately, 200 to 5,000 of the treated cells were plated at T-25 flasks (Falcon, Franklin Lakes, N.J.) and returned to the incubator for colony formation. Fourteen days later the colonies were fixed in ethanol and stained with 1% crystal violet. Colonies that contained more than 50 cells were counted. Survival was calculated as the average number of colonies counted divided by the number of cells plated times the plating efficiency (PE), where plating efficiency was the fraction of colonies counted divided by cells plated without radiation. The clonogenic survival data was generated using Fit v2.4 software (provided by Dr. N. Albright, University of California at San Francisco, San Francisco, Calif.).

### EXAMPLE 15

### Cell Proliferation Assay

SW626 cells were plated in 12-well plates in triplicate. Cells were irradiated with 0 to 8 Gy as above, and 24 hrs later cells were infected with Ad/Bax and Ad/Cre or control virus. Starting 24 hrs later and periodically until 120 hrs post-radiation, groups of cells were trypsynized and counted manually after staining by trypan blue exclusion and in a Coulter counter machine. The corresponding cell numbers were plotted in a cell growth curve.

### EXAMPLE 16

### Apoptosis Assays

Apoptosis was evaluated by determining cell viability, nuclear morphology, and by a quantitative test that measures an early apoptotic plasma membrane alteration. For visualization of nuclear morphology, cells (0.5×10⁶) were stained with the intercalating DNA dye H33258 (Sigma, St. Louis, Mo.), and observed under a UV light microscope. An early apoptotic alteration in the plasma membrane, in the form of translocation of phosphatidylserine (PS) from the inner side of the plasma membrane to the outer layer, was also quantified by a fluorescence-activated cell sorter (FACS) using the Annexin-V-FLUOS Staining Kit (Boeringer Mannheim, Indianapolis, Ind.), as per the manufacturer recommendations. The proportion of cells stained by propidium iodide, Annexin V, or both, was determined using an Epics Coulter Cytometer.

### EXAMPLE 17

### Immunoblot Analysis

Cells were lysed with a NP-40 buffer containing 0.3% NP-40, 142 mM KCl, 5 mM MgCl₂, 2 mM EDTA, 20 mM HEPES pH7.4, and a cocktail of protease inhibitors. Total protein (10-50 μg) was separated on a 12% SDS-polyacrylamide gel and subsequently transferred to a PVDF membrane (BIO-Rad, Hercules, Calif.). The membranes were blocked with 2% non-fat dry milk and probed with a HA monoclonal antibody at a dilution of 1:1,000 (Santa Cruz Biotechnology, Santa Cruz, Calif.). After washing with TBS-Tween (50 mM Tris-HCl, pH 8.3, 150 mM NaCl, and 0.5% Tween-20), an HRP conjugate secondary antibody was added, followed by detection with an enhanced chemoluminescence system (Renaissance, NEN, Boston, Mass.).

### EXAMPLE 18

### In vivo Tumor Nodule Formation Experiment

In this experiment, nude mice 4 to 6 week old were used. Animals had been classified in 5 groups (n=5), according to cell treatment as follows: radiation alone, irrelevant virus alone or with radiation (8 Gy), and Ad/Bax and Ad/Cre alone or with radiation (8 Gy). Animals were injected subcutaneously into both upper flanks with 20×106 SKOV3ip1 cells, and then followed for nodule formation every other day for up to 8 weeks.

### EXAMPLE 19

### Generation of an Inducible Recombinant Adenoviral Vector Encoding Bax using the Cre-loxP System

Overexpression of pro-apoptotic molecules such as Bax is known to be cytotoxic in many cell lines. To generate a recombinant adenoviral vector encoding Bax (Ad/Bax), an inducible vector based in the Cre-loxP system was employed. In this system, the bax gene cannot be translated until the loxP-neo^{r}-loxP cassette is excised by Cre recombinase. Thus, Bax would not be expressed in vector-producing cells or other adenovirus-infected cells unless Cre is coadministered (FIG. 5A). Expression of Bax protein was confirmed by coinfection of ovarian cancer cells with Ad/Bax plus Ad/Cre followed by immunoblotting analysis with an anti-HA monoclonal antibody (FIG. 5B). Thus, in the context of adenoviral-mediated gene transfer, expression of Bax can be tightly controlled upon induction with Cre.

### EXAMPLE 20

### The Recombinant Adenovirus Ad/Bax Radiosensitizes Ovarian Cancer Cell Lines

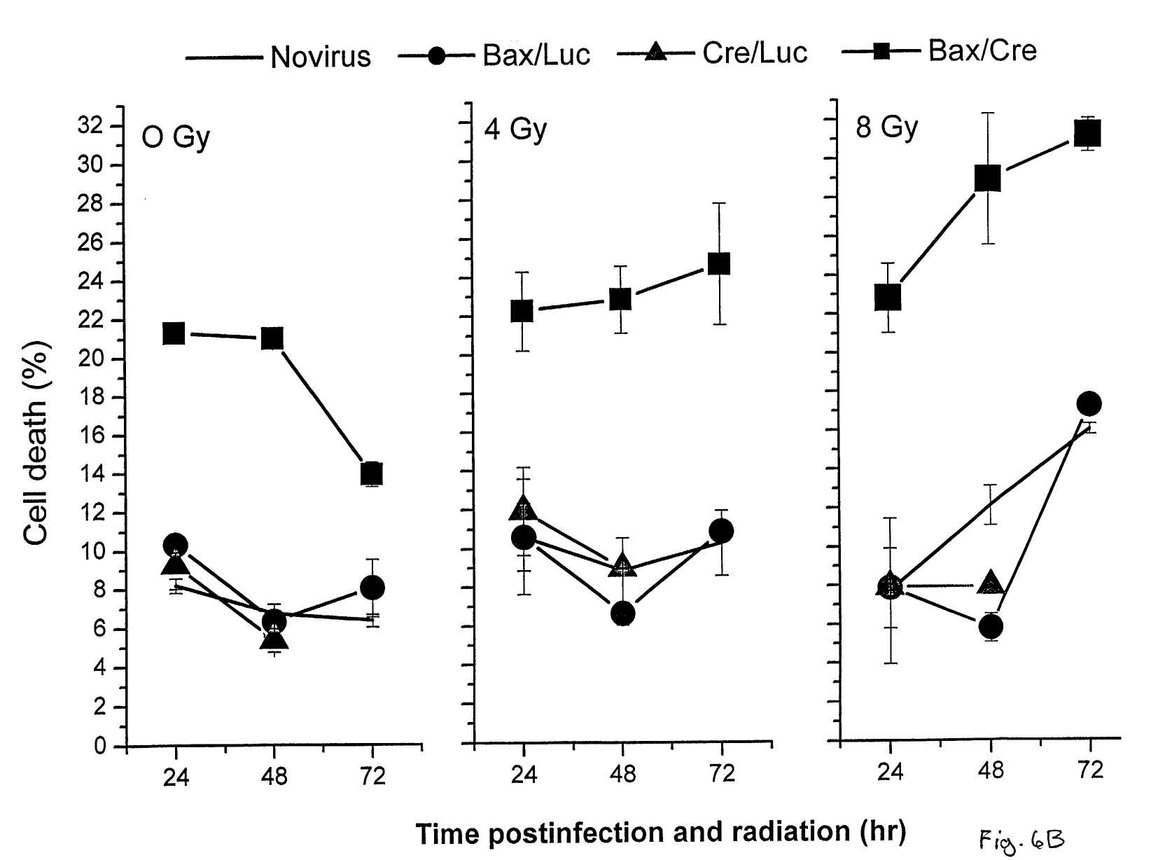

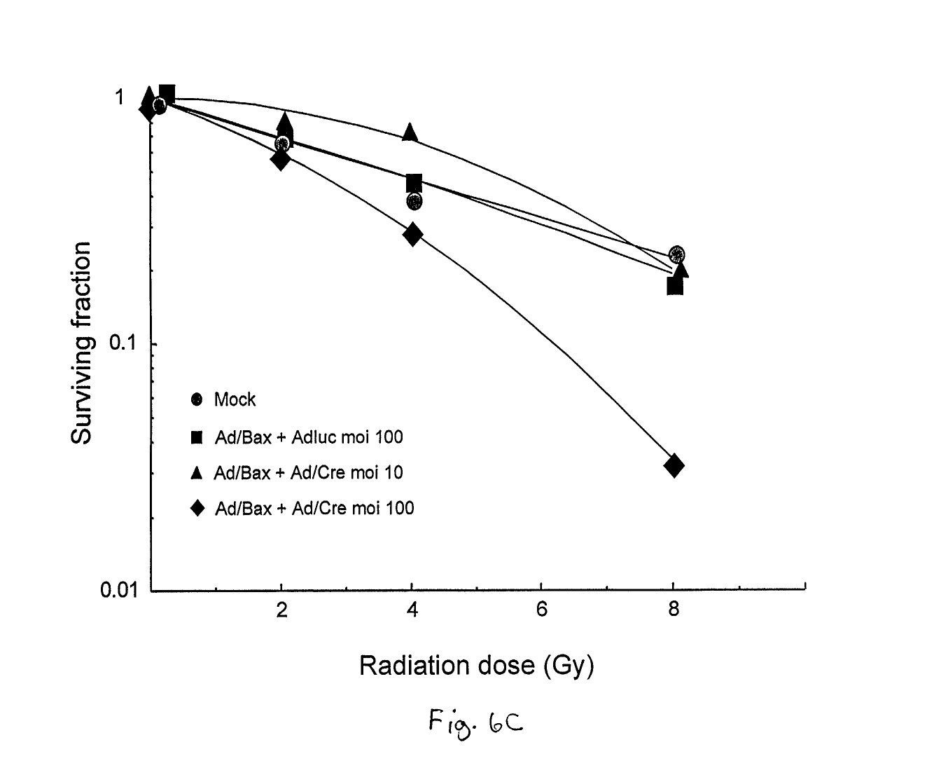

To evaluate the ability of Bax expression to sensitize refractory cancer cells to radiation, the human ovarian cancer cell line SKOV3.ip1, which is known to be significantly resistant to radiation and has been used by several groups, was assayed in well-characterized in vivo models of ovarian cancer and SW626, which has previously been tested for Bax sensitivity. As an initial test, SKOV3.ip1 cells was infected with Ad/Bax, with or without the inducer vector Ad/Cre, and then irradiated the cells with several doses of ionizing radiation, ranging from 0 to 8 Gy. For SW626, cells were irradiated with 0 or 8 Gy, and transduced 24 hrs later at an MOI of 100. After 5 days, a cell viability assay was performed by staining the remaining cells with crystal violet, showing that there were significantly higher levels of cell killing in the combined viral plus radiation treatment group than in the control groups (FIG. 6A). Importantly, a similar biological effect on primary cells derived from patient ascites (FIG. 2A) was observed.

To evaluate in a quantitative manner the levels and time course of the killing effect, cell death was measured by FACS, as determined by cell staining with the necrosis marker propidium iodide and the early apoptotic marker Annexin V. When Ad/Bax was administered with Ad/Cre, levels of cell death at 24 hr post-infection were more than twice the levels observed after treatment with Ad/Bax and irrelevant Ad/luc, or after no treatment (both, p<0.00001). However, the induction of cell death with Ad/Bax plus Ad/Cre decreased noticeably at 72 hr post-infection, falling from 21.3 to 13.9±0.6% (FIG. 6B). Importantly, combined treatment of Ad/Bax, Ad/Cre and radiation increased the levels of cell death in a dose and time-dependent manner. For example, after radiation with 8 Gy, cell killing increased from 22.8±1.7% at 24 hr to 31.1±0.8% at 72 hr (FIG. 6B). In contrast, radiation alone induced at this dose cell killing of 7.7±3.7% at 24 hr and of 16±0.2% at 72 hr. At both time points, the increase of cell death induced by the combined treatment versus radiation alone was statistically significant (p<0.00001). Thus, treatment with Ad/Bax and Ad/Cre was able to increase between two to three fold the magnitude of in vitro cell killing after radiation in this partially radiation-refractory ovarian cancer cell line, and this effect augmented with time.

As an additional standard test for radiosensitization of human ovarian cancer cells genetically modified with Bax, an in vitro assay of colony formation was used. SKOV3.ip1 cells were treated with a range of radiation doses, infected, and then plated for colony formation. As shown in FIG. 6C, there was a dramatic difference in survival between SKOV3.ip1 cells which had been treated with Ad/Bax and Ad/Cre following exposure to varying doses of radiation (between 0 and 8 Gy) and the other groups, including radiation alone, radiation with 10 pfu of Ad/Bax and an irrelevant virus, or radiation with 100 pfu of Ad/Bax and Ad/Cre. In effect, a reduction of 98% of colonies was observed after combined treatment with the highest dose of virus and radiation, whereas only 48% cells were killed when they received radiation or the bax gene alone. The D₀, a standard radiobiological parameter determined by the dose that keeps alive 37% of colonies tested, was 4.8 Gy for radiation alone, consistent with the level of radioresistance observed in other epithelial cell lines. In contrast, the D₀ was 2.1 Gy for the combined radiation and Bax treatment at the same doses. These data showed that Bax gene adenoviral-mediated delivery increases cell death in ovarian cancer SKOV3.ip1 cells, and augments the killing effect of ionizing radiation in these previously radiation-refractory cells.

To extend these findings, the effect of Bax was tested in the SW626 cell line. Cells were infected with Ad/Bax and Ad/Cre or irrelevant virus 24 hrs after radiation with 0 to 8 Gy, and then were counted at different time points. Cells that received no treatment or that were treated with irrelevant virus grew to reach 300% of initial cell number after 120 hrs post-radiation. Cells treated with Ad/Bax and Ad/Cre alone experienced a delay in growth during the first 72 hrs, but started to grow immediately after, and reached 150% of the initial number. Cells treated with radiation alone grew steadily to reach 90% of the initial number after 120 hrs. In contrast, cells treated with radiation and Ad/Bax plus Ad/Cre showed a marked inhibition of growth, reducing its number to 18% of the initial number after 120 hrs (FIG. 6D). In radiobiological terms, these results showed a LD₅₀ of 4 Gy for radiation alone and of 1.2 Gy for the combined treatment of radiation plus Bax (data not shown).

### EXAMPLE 21

### Radiosensitization by Ad/Bax Involves Induction of Both Apoptosis and Necrosis

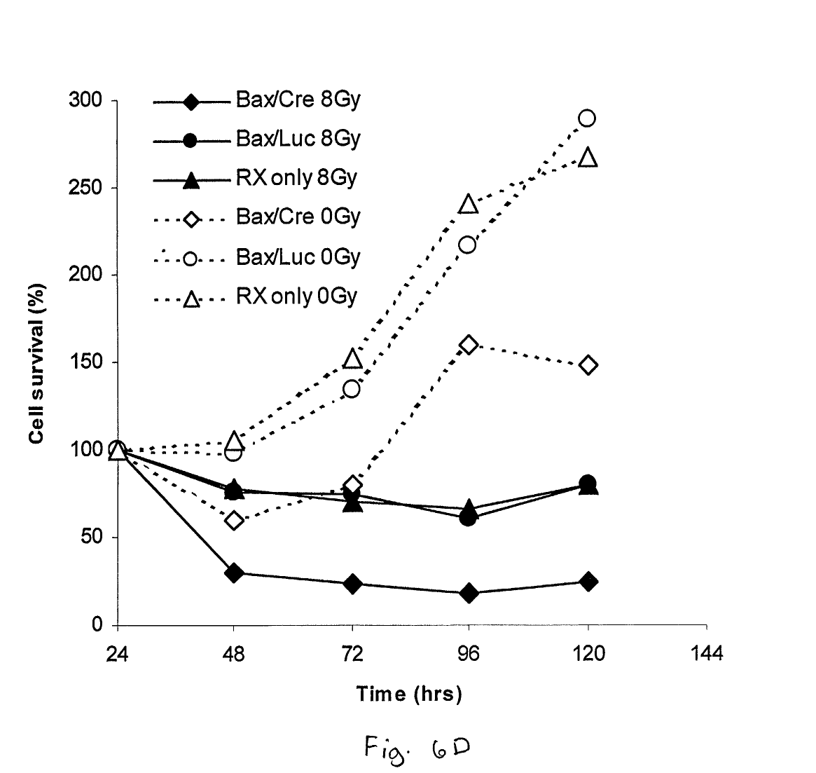

To determine whether Ad/Bax and radiation mediate cell death by facilitating apoptosis, SKOV3.ip1 were coinfected with Ad/Bax plus Ad/Cre, Ad/Cre alone, or Ad/Bax alone in the presence or absence of different doses of radiation. Cell morphology was examined by standard bright field microscopy. Typically, cells coinfected with Ad/Bax plus Ad/Cre and then radiated became rounded, shrank, and subsequently detached from the tissue culture plates and died. Nuclear staining with the intercalating DNA dye Hoechst H33256 revealed that a substantial fraction of infected cells displayed nuclear condensation and fragmentation and mitotic cell death 48 hrs post-radiation, as analyzed by UV microscopy (FIG. 7). On the other hand, neither the group treated with radiation alone or with irrelevant virus plus radiation showed any typical apoptotic effect even with a high dose of radiation. This observation was expected in this cell line, known to be refractory to ionizing radiation. Consistent with this, less frequent phenotypic changes suggestive of apoptosis were identified in the group treated with Ad/Bax and Ad/Cre virus without irradiation.

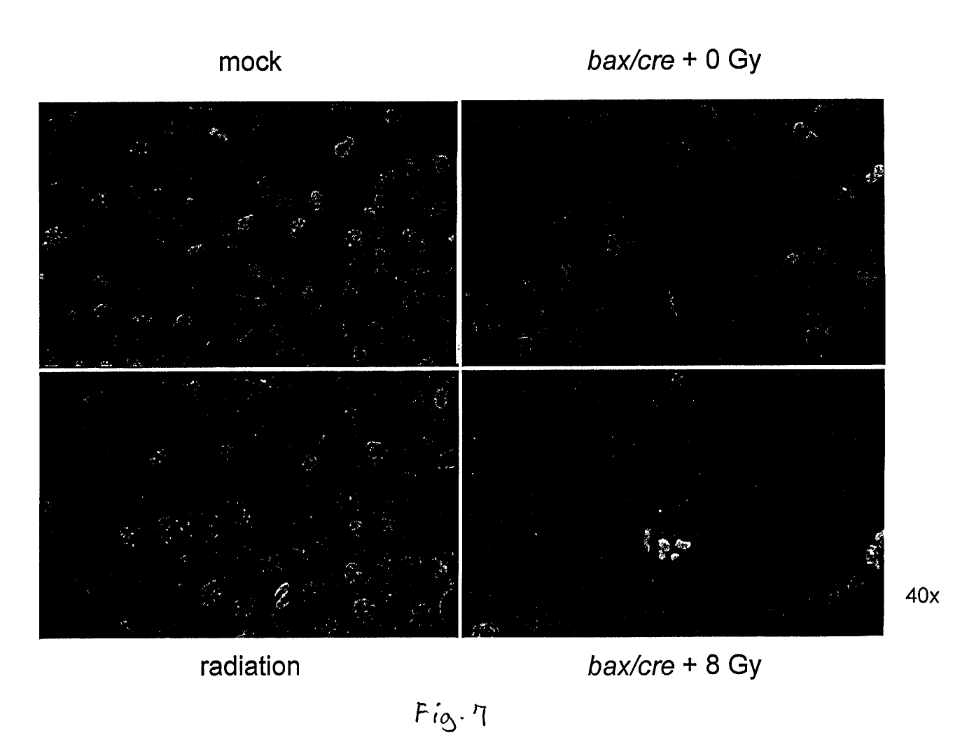

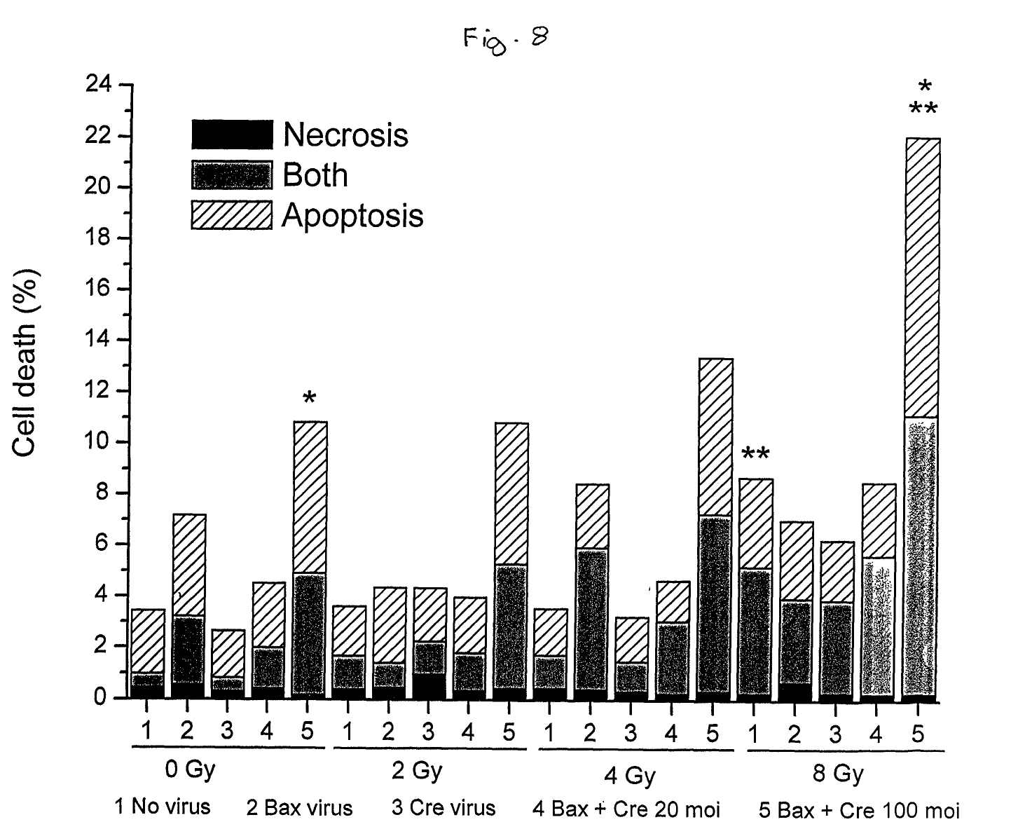

An additional, quantitative assay was performed to directly confirm the mechanism of cell killing measuring by FACS the levels of Annexin-V, which binds apoptosis-specific phosphatidylserine in the outer cell membrane, and propidium iodide, which penetrates and binds cellular DNA in necrotic cells. Firstly, the cell killing induced at 24 hr post-radiation by treatment with Ad/Bax, inducing Ad/Cre, and radiation was dose-dependent (FIG. 8). Secondly, there was a varying degree of apoptosis and necrosis in the different experimental groups, with a trend toward higher levels of apoptosis in cells treated with combined Ad/Bax, inducing Ad/Cre, and radiation. Both the proportion and the absolute number of apoptotic cells was higher in that group, as compared with the rest of experimental groups, including cells treated with an irrelevant virus and radiation, cells treated with radiation alone, and cell treated with virus alone (FIG. 8). Similar statistically significant differences between relevant groups were observed at 72 hr, with even higher absolute levels of cell death (data not shown).

### EXAMPLE 22

### Tumor Nodule Formation in vivo

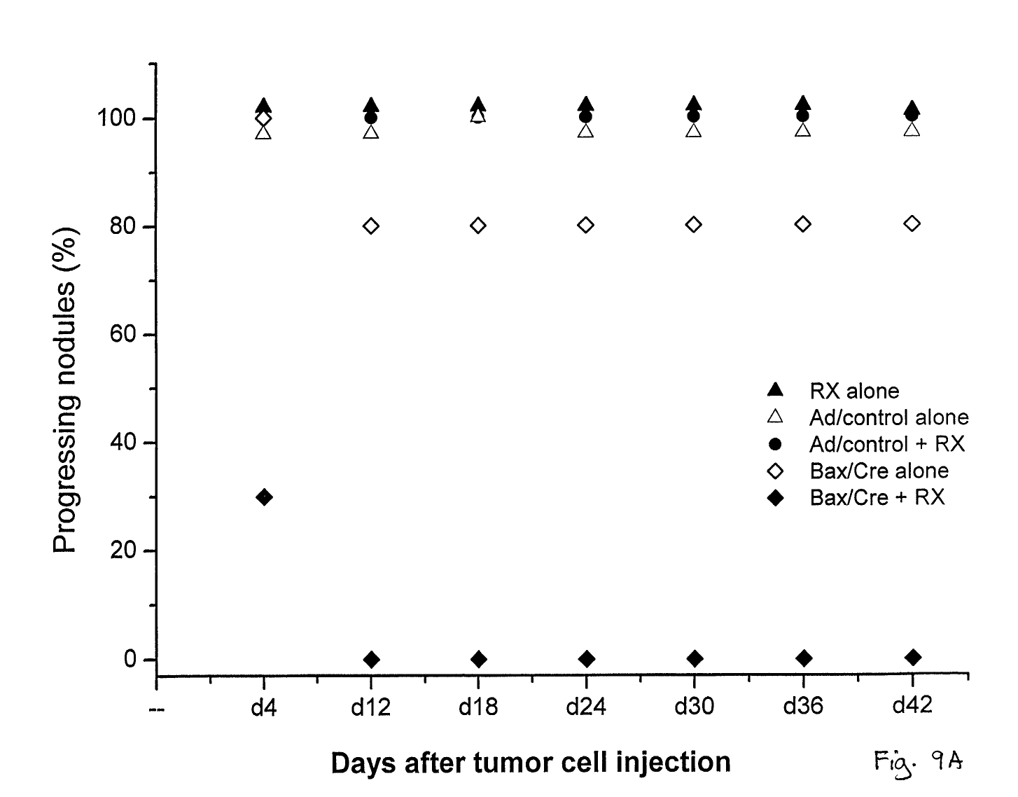

To explore the efficiency of Bax-mediated radiosensitization in vivo, nude mice were injected subcutaneously with ovarian cancer cells treated or not with Ad/Bax and Ad/Cre, or an irrelevant adenovirus (Ad/CD) as control, and then irradiated with 0 Gy or 8 Gy. Animals were then followed for tumor nodule formation. Tumor nodules developed in all animals (2 out of 2 nodules per animal, 10 out of 10 nodules per group) with the exception of those animals injected with cells treated with Ad/Bax and Ad/Cre combined with radiation (FIG. 9). In this group, 3 out of 10 small nodules developed during the first week and quickly disappeared, without any further evidence of tumor growth during the next 8 weeks of observation. Of note, 8 out of 10 nodules developed in the animals receiving cells treated with Ad/Bax and Ad/Cre but non-irradiated. These nodules were smaller, however, compared to the other control groups.

Aberrant expression of normal cellular oncogenes or loss of tumor suppressor gene function can lead to diminished sensitivity of tumors to conventional therapeutic regimens resulting in a more aggressive and refractory phenotype. One novel gene therapy strategy is to restore within a tumor an increased responsiveness to conventional therapy via gene delivery. In this regard, functional restoration of Bax, a pro-apoptotic member of the Bcl-2 family and a putative tumor suppressor, may have utility as a sensitizer of tumor cells to conventional chemotherapy or radiotherapy. In this report significantly enhanced ovarian tumor cell killing was demonstrated when radiation is administered in combination with Ad/Bax compared to either radiation or Ad/Bax alone. Moreover, the increased sensitivity of tumor cells is both radiation and virus dose-dependent, and the killing effect of Bax in vitro is prolonged by virtue of its interaction with radiation. Additionally, consistent with the proposed mechanism of Bax mediated cell death, a significant degree of apoptosis is observed in ovarian carcinoma cells exposed to Ad/Bax and radiation. Moreover, this in vitro data has been successfully translated into a significant radiosensitization of ovarian carcinoma in an in vivo model.

These findings are of particular interest as the experiments were performed using an ovarian carcinoma cell line. Ovarian carcinoma represents an ideal tumor target for the study of gene therapy as a sensitizing adjunct to conventional therapy. Specifically, ovarian carcinoma is a tumor most often confined to the peritoneal cavity and accessible for intraperitoneal treatments based on gene delivery. Additionally, ovarian carcinoma is responsive to conventional therapy with chemotherapy or radiotherapy although durable cures are rare. Ovarian tumor cell lines and primary tumors have demonstrated a substantial rate of gene transduction using adenoviral vectors. Finally, advances in molecular biology have allowed for the identification of genetic lesions associated with ovarian carcinoma that can be targeted by gene therapy approaches. To this end, alterations in ovarian carcinoma of the pro-apoptotic gene bax have led to investigations targeting this gene.

The ability of alterations in expression of Bax to modify the response to chemotherapeutic agents provided a compelling rationale to explore sensitization mediated by Bax in the context of radiation. Radiation induces up-regulation of Bax and apoptosis in vivo in radiosensitive tissues. Studies examining Bax as a radiosensitizer are particularly relevant in light of the data by Strobel et al. suggesting that sufficient levels of transfected Bax could bypass the need for upstream molecules, such as p53. SKOV-3.ip1 and SW626 cells were used in the current study, which are known to be deficient in p53. It is also known that Bax can function downstream of p53 signal pathways. In effect, the expression of Bax enhanced sensitivity of SKOV3.ip1 cells to radiation, supporting the concept that the need for upstream regulators of apoptosis such as p53 can be overcome. These results support further investigations in other cell lines with variable expression of p53 and other upstream modulators of apoptosis.

The finding in this study that the mechanisms of cell death are both apoptosis and necrosis supports the concept that overexpression of Bax can mediate programmed cell death and necrosis by caspase-dependent and caspase-independent pathways, respectively. These results are in accord with, and extend with a more relevant cellular substrate, previous studies of radiosensitization performed in stable clones of the breast cancer cell line MCF-7 expressing Bax. These last findings are in contrast to an investigation of radiosensitization using ovarian cancer SW626 cell clones stably expressing Bax (Strobel, et al., Oncogene. 1997;14:2753-2758). This cell line was used in the transient model of Bax expression, and, in contrast, showed a significant radiosensitizing effect of Bax. This difference might be due to the recognized complex set of phenomena associated with these cells being stably transfected. These authors were not able to demonstrate induction of apoptosis in response to ionizing damage. However, one critical difference between both studies is the use of an inducible system to transiently augment Bax expression for a therapeutic advantage. The enhanced effect of the radiation is over and above that seen with bax transfection alone. Also, these results were obtained in a more heterogeneous cellular population than that represented by a particular stable clone, unavoidably selected for an uncontrolled number of genetic traits.

The results of this study demonstrate a radiosensitizing effect of Bax expression in two variably refractory ovarian carcinoma cell lines after gene delivery via recombinant adenovirus. Comparable results were also obtained with primary, patient-derived cancer cells and in an in vivo murine xenograft model of ovarian carcinoma. These results suggest that the combination of Ad/Bax and radiation therapy might have clinical utility.

### EXAMPLE 23

### Cell Lines and Culture Conditions

The 293 human kidney cell line and human ovarian cancer cell lines OVCAR3, PA-1, and SW626 were purchased from the American Type Culture Collection (ATCC, Rockville, Md.). The ovarian cancer cell line SKOV3.ip1 was from Dr. Janet Price (M. D. Anderson Cancer Center, Houston, Tex.). The human ovarian carcinoma cell line OV-4 was from Timothy J. Eberleim (Brigham and Women's Hospital, Harvard Medical School, Boston, Mass.). All cell lines were maintained in Dulbecco's Modified Eagle's Medium:F-12 (50:50 mixture) containing 10% of fetal bovine serum (FBS), except OVCAR3, which is cultured in RPMI-1640 with 10% of FBS and 10 μg/ml insulin.

### EXAMPLE 24

### Purification of Primary Ovarian Cancer Cells

Primary ovarian cancer cells were isolated by affinity purification from ascites obtained from patients with ovarian cancer. Fresh ascites was centrifuged to collect cells in suspension. The cells were then washed and resuspended in PBS with 0.1% BSA. The CC49 antibody (provided by Dr. Jeffrey Schlom, NIH, Bethesda, Md.), which specifically recognizes the pancarcinomatous antigen TAG-72, was added to the cell suspension at a concentration of 1 μg/10⁷ cells, and incubated at 4° C. for one hour. To isolate the immuno-complex formed by the CC49 antibody and the TAG-72 expressing tumor cells, magnetic Dynabeads (Dynal Inc. Lake success, N.Y.) coated with a secondary anti-panmouse IgG antibody were added at a ratio of 4 beads/cell, according to the protocol of the manufacturer. The bead-targeted complexes were then washed and captured with a magnetic particle concentrator (Dynal Inc.). Finally, the purified cells were released from the beads by brief trypsinization and recovered in RPMI complete medium. Pathological analysis of these purified cells showed that over 95% of the cells were ovarian carcinoma cells.

### EXAMPLE 25

### Recombinant Adenoviruses and Infection of Cells

The replication-incompetent adenoviral type 5 recombinant vectors encoding bax (Ad/Bax), and Cre recombinase (Ad/Cre), were generated as described. Ad/LacZ, encoding the E. coli LacZ gene, was provided by Robert Gerald (The Center for Transgene Technology and Gene Therapy, Leuven. Belgium). All recombinant adenoviruses were propagated in 293 cells and purified by centrifugation in CsCl gradients by a standard protocol. For viral infection in dishes, the cells were plated in 6 or 96 wells the day before the experiment. The viruses were then added at a designed multiplicity of infection (moi) and incubated for 1 to 2 hours. For infection in suspension, about 2×10⁶ cells /ml were incubated with viruses for 1 to 2 hours. The ratio of Ad/Bax:Ad/Cre was 5:1 at a total moi of 100, unless indicated otherwise. After infection, the cells were washed and maintained in culture, with or without various concentration of taxol, for additional 48 hours.

### EXAMPLE 26

### Cytotoxicity Assay and Apoptosis Assay

Cells were seeded in either 6-well or 96-well plates 24 hours before viral infection. The cells were then infected with either Ad/Bax plus Ad/Cre or control viruses for 1 hour. The cells were washed and then cultured in complete medium with or without taxol (MeadJohnson, Princeton, N.J.) at variety of concentration ranges for an additional 48 hours. Cell viability was determined using a calorimetric method with 3-(4,5-dimehtylthiazol-2-yl)-5-(3-carboxymethoxyphenyl)-2-(4-sulfophenyl)-2H-tetrazolium (MTS) according to the instructions of the manufacturer (Promega). The IC₅₀ values, defined as the drug concentration that reduced the absorbance by 50% or as a 50% cell kill relevant to untreated cells, were estimated graphically from the concentration-response curves. For visualizing apoptotic cells, the cells were fixed with methanol and then stained with H33258, and visualized by UV microscopy.

### EXAMPLE 27

### Immunoblotting

Cells were lysed in an NP-40 lysis buffer containing 142 mM KCl, 5 mM MgCl₂, 2 mM EDTA, 2 mM EGTA, 0.3% NP-40, and 20 mM HEPES pH 7.4. Total protein from cell lysates (50 μg per lane) were resolved by SDS-polyacrylamide gel electrophoresis, followed by transfer onto a 0.2 μm polyvinylidene difluoride (PVDF) membrane (Bio-Rad). The membrane was then blocked for one hour in Tris-buffered saline (TBS) with 2% non-fat milk. The blots were subsequently probed with an HA monoclonal antibody at a dilution of 1:800 (Santa Cruz Biotechnology, Santa Cruz, Calif.), an anti-Bcl-2 antibody at a dilution of 1:2000 (DAKO, Carpinteria, Calif.), an anti-p53 monoclonal antibody at a dilution of 1:1000 (Santa Cruz), or an anti-Bax polyclonal antibody at a dilution of 1:1000 (Santa Cruz). After washing with TBS containing 0.05% Tween-20, a secondary antibody conjugated to horseradish peroxidase (Jackson 1:3000 diluted in TBST) was added for a one-hour incubation. Finally, the blot was washed and developed using the enhanced chemiluminescence (ECL) detection system (Amersham) according to the manufacturer's protocol and exposed to an radiographic film (Eastman Kodak).

### EXAMPLE 28

### Inducible Adenoviral Vector-mediated bax Gene Transfer Results in Tight Control of Bax Expression in Ovarian Cancer Cell Lines

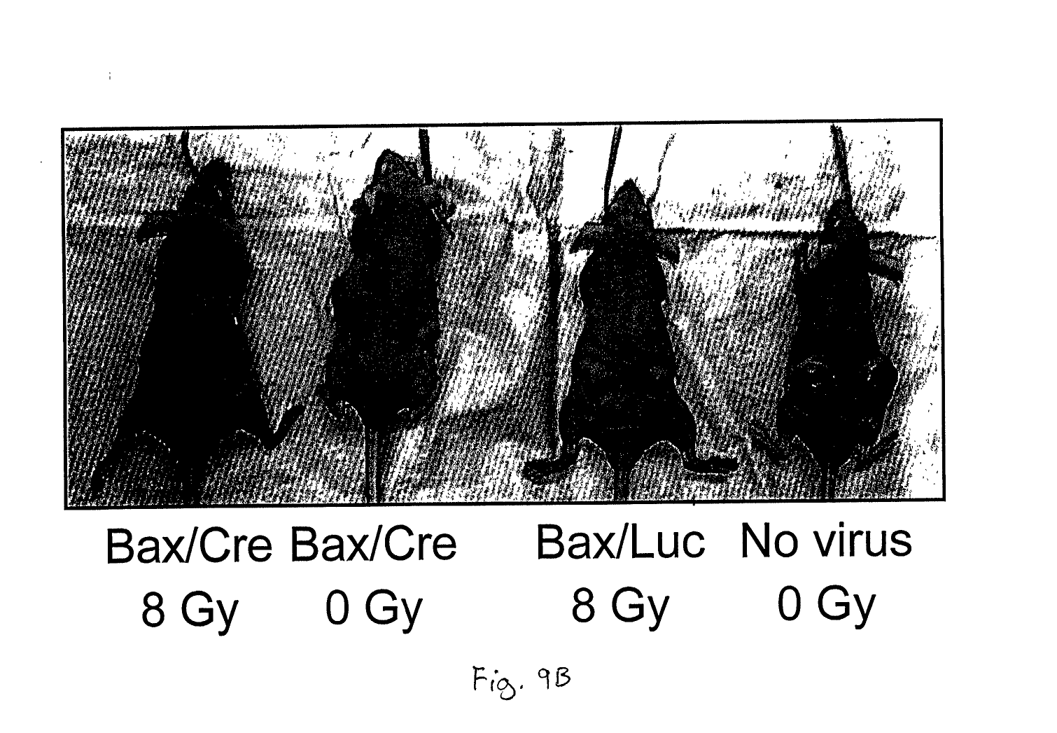

To deliver the pro-apoptotic bax gene into ovarian cancer cells, a recombinant adenoviral vector encoding bax was generated using the LoxP/Cre inducible system (FIG. 10A). The advantage of this system is that there is no expression of Bax protein in the absence of Cre recombinase. Therefore, this type of control allows the propagation of the recombinant Bax adenovirus by avoiding the cytotoxicity of Bax to the host 293 cells. Expression of the Bax protein can be induced in the target cells by co-infection with a recombinant adenovirus encoding Cre recombinase (Ad/Cre) (FIG. 10A). Using this delivery system, a panel of ovarian cancer cell lines was screened. As shown in FIG. 10B, Bax expression was dramatically induced in all cell lines infected with Ad/Bax plus Ad/Cre. In contrast, there was no significant Bax expression when Ad/Bax was co-infected with the control virus Ad/LacZ (FIG. 10B). Bax induction could be detected as early as 8 hours, and peaked by 24 to 48 hours post-infection. These results suggest that the LoxP/Cre system provides a sufficient and tightly controllable means to mediate expression of the pro-apoptotic gene Bax through viral gene delivery into various ovarian cancer cell lines.

### EXAMPLE 29

### Ovarian Cell Lines Display Differential Cytotoxic Sensitivities to Bax Mediated Cell Death

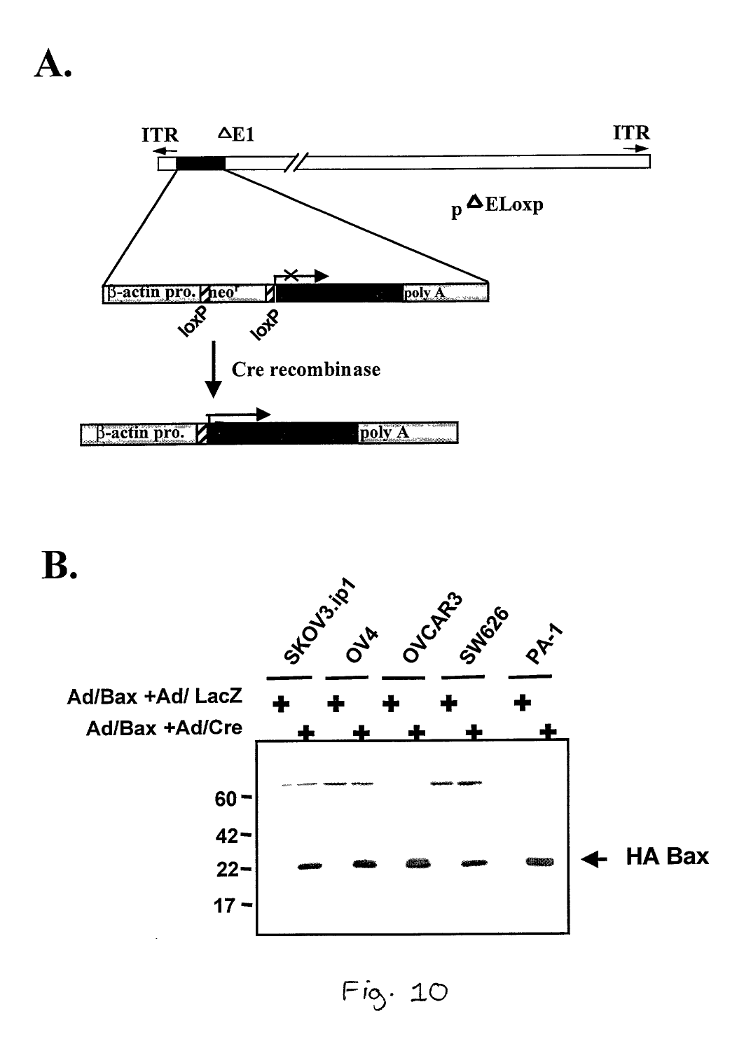

To determine the cytotoxic effects of Bax, five ovarian cancer cell lines were co-infected with Ad/Bax and Ad/Cre or Ad/lacZ and Ad/Cre at ratio of 5 to 1 at a total moi of 100. After 48 hours, cell viability was determined by MTS assays (FIG. 11). Induction of Bax expression caused cell death in all tested ovarian cell lines, though the sensitivities of each cell line to Bax-mediated cell death varied. For OVCAR3, OV4, and PA-1 cells, over 80-90% of cells were killed. Under the same conditions, there was only 40 to 60% of cellular death with SKOV3.ip1 and SW626 cells (FIG. 11), which also showed resistance to even higher moi (300) of Ad/Bax. This differential sensitivity is not due to differences in the infectability of the adenovirus. For example, SKOV3.ip1 and OVCAR3 cells can be infected at similar levels (80-90%) with adenovirus (data not shown), but SKOV3.ip1 cells were much more resistant to Bax-mediated cell death than OVCAR3 cells (FIG. 11). The death of the ovarian cancer cells examined had typical apoptotic features, including nuclear condensation, fragmentation, and positive staining with Annexin V. These data indicate that different ovarian cancer cell lines have heterogeneous apoptotic thresholds to Bax-mediated cell death.

### EXAMPLE 30

### Bax-mediated Cell Death in Ovarian Cancer Cells is Independent of the Endogenous Levels of Bcl-2 and Bax Proteins and Cellular p53 Status

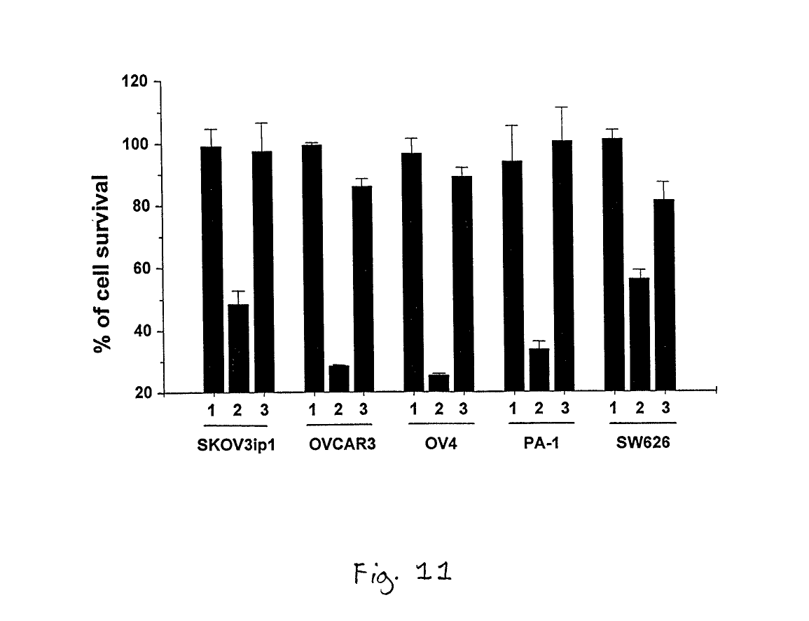

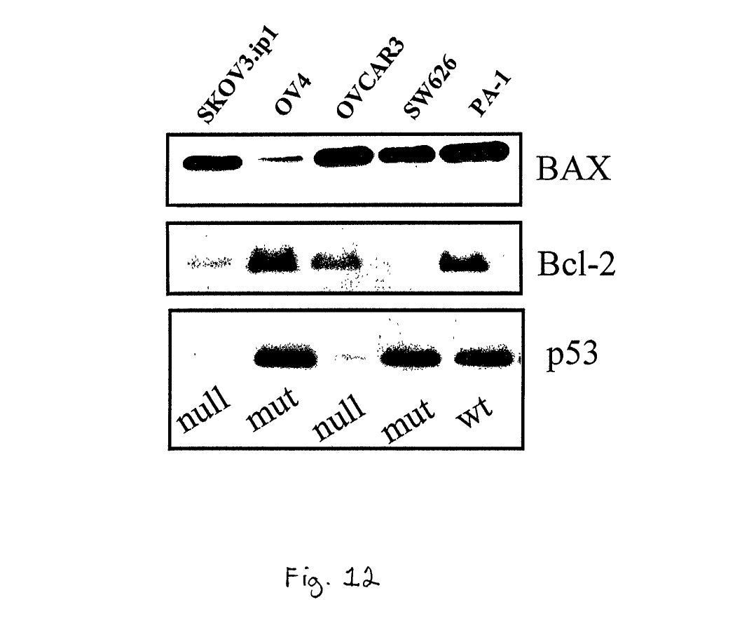

Bax promotes apoptosis through formation of heterodimers with the anti-apoptotic molecule Bcl-2. In addition, Bax serves as a downstream target in p53-mediated apoptotic pathway. Therefore whether Bax-mediated apoptosis in these ovarian cancer cell lines is dependent on the endogenous levels of Bcl-2 and Bax or the p53 status was tested. As shown in FIG. 12, the levels of Bcl-2 and Bax varied among different ovarian cell lines. There was no correlation between the degree of Bax-mediated killing and the endogenous levels of Bcl-2 and Bax. The bax-mediated cell killing also appeared to be independent of the status of p53. A similar percentage of cells were killed in the cells with wild-type p53 (PA-1), or mutant or null p53 (OV4 and OVCAR3) (FIGS. 11 and 12). These results are in agreement with previous reports suggesting that Bax is able to induce cell death in a Bcl-2 and p53 independent manner. Importantly, these results indicate that the use of Bax as a therapeutic agent for tumors that are composed of heterogeneous cell populations is feasible.

### EXAMPLE 31

### Adenoviral-mediated bax Gene Delivery Leads to Apoptosis in Primary Ovarian Cancer Cells

To test Bax-mediated killing in primary ovarian cancer cells, primary cells from ascites of ovarian cancer patients was purified using the adenocarcinoma-specific antibody CC49. Over 90% of the purified cells were typical ovarian carcinoma cells, as revealed by morphological analysis and immunochemical staining for tumor-specific markers. The purified cells were co-infected with Ad/Bax and Ad/Cre or Ad/LacZ and Ad/Cre at a ratio of 5 to 1 and a total moi of 500 (FIG. 13A). The cells were collected 48 hour post-infection. Expression of Bax protein was determined by immunoblot analysis with an anti-HA antibody which only detects HA-tagged Bax (FIG. 13A), while cell viability was analyzed by MTS assay. The Bax expression pattern was similar to that of the ovarian cancer cell lines tested (FIG. 10B). Co-infection of Ad/Bax and Ad/Cre led to detachment of a majority of cells from the culture dishes by 24 to 48 hours. Nuclear staining with H33256 revealed that the cells contained condensed, fragmented nuclei, and apoptotic bodies characteristic of apoptosis (FIG. 13B). Under the same conditions, the control cells, which were infected with Ad/LacZ and Ad/Cre, had no significant apoptotic morphological changes (FIG. 13B). This result strongly suggests that Bax-mediated cell death is an ubiquitous phenomenon, and is not associated with specific tumor cell lines or clonalization.

### EXAMPLE 32

### Adenoviral-mediated bax Gene Transfer Enhances the Cytotoxic Effect of Chemotherapy in Ovarian Cancer Cell Lines

Stable overexpression of Bax in some cancer cell lines synergizes the killing effect of chemotherapy or radiotherapy. To test whether adenoviral-mediated bax gene transfer can produce the same synergy, two ovarian cell lines resistant to Bax, SW626 and SKOV3.ip1 were treated with the chemotherapeutic drug taxol, alone or in combination with Ad/Bax plus Ad/Cre or Ad/LacZ plus Ad/Cre (FIG. 14). Cell viability was measured by an MTS assay. Overexpression of Bax synergized the cytotoxicity of taxol in both cell lines (FIG. 14). In the control cells, the IC₅₀ of taxol was 31 μg/ml in SW626 cells and 39 μg/ml in SKOV3.ip1 cells. After combined treatment with Bax, the corresponding IC₅₀ of taxol were 5 μg/ml and 13 μg/ml, respectively (FIG. 14). This synergy was not the result of adenovirus infection, since co-infection with control viruses did not enhance the cytotoxicity of taxol (FIG. 14). These results suggest that Ad/Bax can indeed enhance the cytotoxic effect of chemotherapy in otherwise refractory ovarian cancer cells.

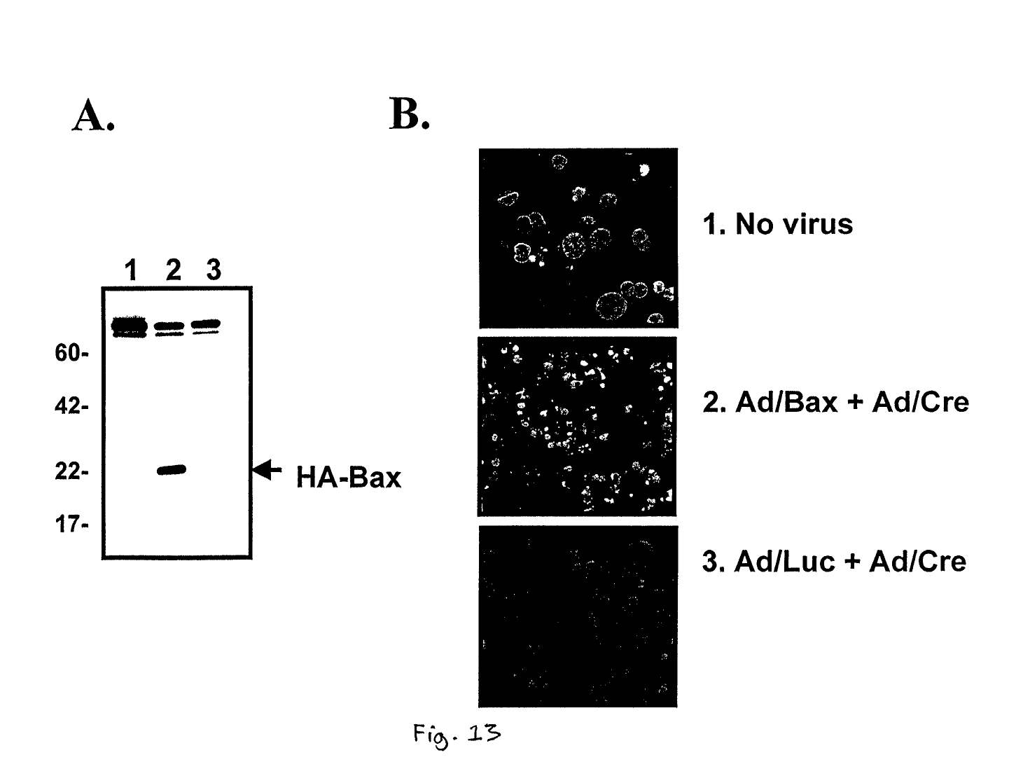

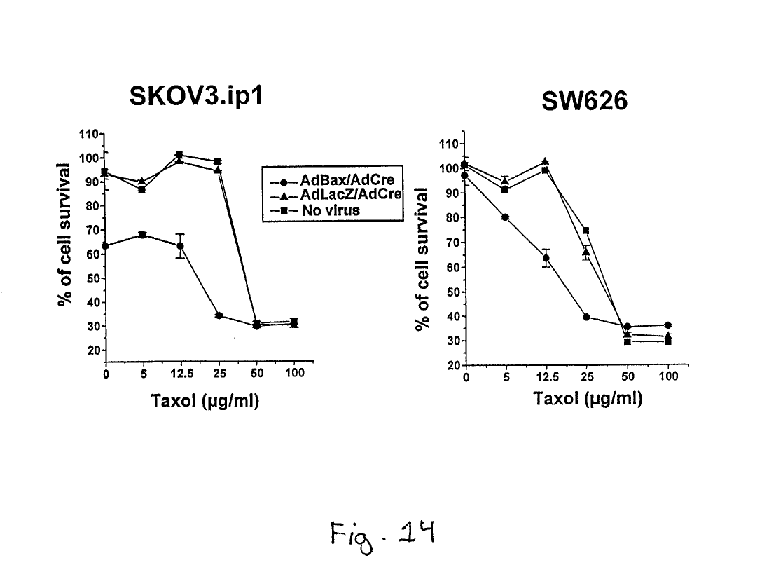

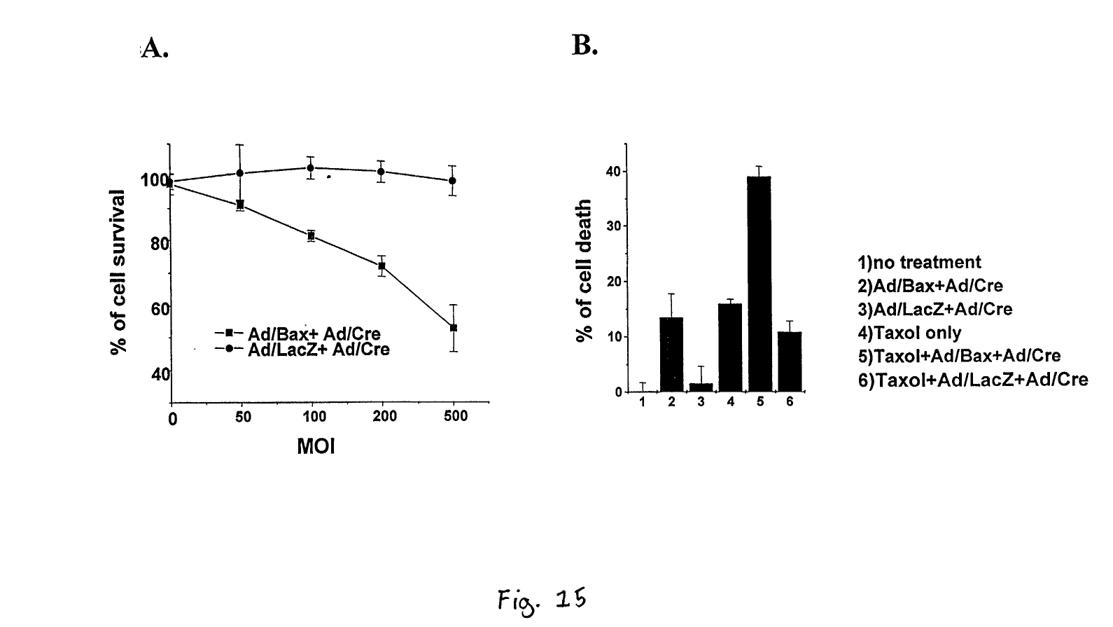

To extend the validity of these findings into a more clinically relevant model system, whether Bax synergizes the cytotoxic effects of chemotherapy in primary ovarian cancer cells was tested. To this end, primary ovarian cancer cells were purified from ascites of ovarian cancer patients using the tumor-specific antibody CC49. After recovery, the purified primary cells were infected with Ad/Bax and Ad/Cre or with the control viruses Ad/LacZ and Ad/Cre at different moi (FIG. 15A), or a constant amount of virus (200 moi) in combination with taxol (FIG. 15B). Expression of Bax alone induced cell death in a viral dose-dependent manner, as measured by MTS assay (FIG. 15A). In a typical experiment, treatment with taxol (20 μg/ml) alone caused 15% cell death. In contrast, over 50% of cell death was observed after combined treatment with Bax. A similar synergistic effect was observed with material from two other patients. Of note, the levels of viral infection in the primary ovarian cancer cells seems to be lower than those obtained in the established ovarian cancer cell lines, since 500 or higher moi of virus had to be used in order to attain infection of 70 to 90% of cells. Nevertheless, the fact that the cytotoxicity of Bax and chemotherapy are synergistic in both primary and established cell lines of ovarian cancer suggests that the effect of Bax is present among all ovarian carcinoma cells of epithelial origin.

In this study, expression of the pro-apoptotic molecule Bax via adenoviral-mediated gene transfer induced apoptosis in various human ovarian carcinoma cell lines as well as primary ovarian cancer cells, although the apoptotic threshold to Bax-mediated cell death is heterogeneous. Furthermore, Bax-mediated apoptosis appears to be independent of the status of p53 and of the endogenous levels of Bcl-2 or Bax. Finally, Bax can synergize the cytotoxic effect of chemotherapy. These results demonstrate the feasibility of utilizing the expression of Bax, alone or in combination with chemotherapy, for cancer gene therapy.

Disturbance of programmed cell death contributes to carcinogenesis, and its manipulation might have therapeutic value. To this end, a pro-apoptotic effect could be induced, in the context of gene therapy for cancer, either through mutation compensation or through direct modulation of the apoptotic pathway in tumor cells. For mutation compensation, restoration of wild-type p53 in tumor cells has been shown to augment the function of the deficient tumor suppressor gene. Alternatively, intracellular expression of single-chain antibodies against the Bcl-2 and erbB2 oncoproteins have proved to be, by ablating the function of those aberrantly expressed dominant oncogenes, a powerful maneuver for cancer therapy.

With the increasing recognition of the molecular basis of the apoptosis pathway, further attempts to explore the therapeutic modulation of the apoptotic pathway in cancer by gene transfer have begun. In this regard, pro-apoptotic molecules, such as Bax, Bcl-Xs, apoptin, and TRAIL all selectively induce cell death in tumors, but not in normal cells. Although the basis for the tumor selectivity of these pro-apoptotic molecules is not totally clear, the feasibility for using these molecules in cancer therapy, allowed by their wide therapeutic index, is very promising. Interestingly, the increase in pro-apoptosis proteins mediated by gene transfer when combined with conventional chemotherapy has a well-documented synergistic effect. This might well be the result of adding a second pro-apoptotic insult, induced by chemotherapeutic drugs, into the signaling pathways that control apoptosis.

Indeed, in stable cell lines, overexpression of Bax induces cell death and can also sensitize the cancer cells to chemotherapy. However, it is not clear whether the effect of Bax is modifed during the process of clonalization. In addition, whether the synergy between Bax and chemotherapy depends on the level of Bcl-2 or the status of p53 is unknown. As a counterpart of Bax, Bcl-2 is critical in determining the balance between cell survival and death. On the other hand, Bax can act as a target in p53-mediated apoptosis. In this report, in both established cell lines and primary patient-derived ovarian cancer cells overexpression of Bax by itself induced apoptosis. The effect of Bax appears to be independent of the level of Bcl-2 or the status of p53. In addition, overexpression of Bax also synergizes with the cytotoxic effect of chemotherapy in primary ovarian cancer cells. It appears that Bax can induce apoptosis in ovarian cancer cells regardless of their heterogeneous genetic background. Thus, overexpression of Bax may provide a solution to the problem of the polymorphism of ovarian cancer and thus enhance the efficacy of chemotherapy.

The mechanism by which Bax synergizes with the cytotoxic effect of chemotherapy has yet to be determined. It has been shown that Bax is able to enhance intracellular levels of taxol. Thus, it would be interesting to study the synergistic regulation mechanism among Bax, bcl-2 and taxol. In any case, it seems feasible to use Bax as a therapeutic agent to enhance the cytotoxicity of chemotherapy. This ability is predicated upon the ability to exert its effects in heterogeneous tumor cell populations with different drug sensitivity profiles.

The following references were cited herein:

1. Mazars, et al., Oncogene. 6: 1685-90, 1991.

2. Santoso, et al., Gynecol. Oncol. 59: 171-8, 1995.

3. Oltvai, et al., Cell. 74: 609-19, 1993.

4. Knudson, et al., Nat Genet. 16: 358-63, 1997.

5. Xiang, et al., Proc. Natl. Acad. Sci. USA. 93: 14559-63, 1996.

6. Schlesinger, et al., Proc. Natl. Acad. Sci. USA. 94: 11357-62, 1997.

7. Yin, et al., Nature. 385: 637-40, 1997.

8. Knudson, et al., Science. 270: 96-9, 1995.

9. Ouyang, et al., Clin Cancer Res. 4: 1071-4, 1998.

10. Yagi, et al., Gastroenterology. 114: 268-74, 1998.

11. Connell, et al., Cell. 34: 245-53, 1983.

12. Parks, et al., Proc. Natl. Acad. Sci. USA. 93: 13565-70, 1996.

13. Zhang, et al., J. Virol. 72: 2483-90, 1998.

14. Kanegae, et al., Nucleic Acids Res. 23: 3816-21, 1995.

15. Rancourt, et al., Clin. Cancer Res. 4: 265-70, 1998.

16. Boente, et al., Curr. Opin. Oncol. 5: 900-7, 1993.

17. Catterall, et al., Glycoconj. J. 14: 867-9, 1997.

18. Clarke, et al., Proc. Natl. Acad. Sci. USA. 92: 11024-8, 1995.

19. Jurgensmeier, et al., Proc. Natl. Acad. Sci. USA. 95: 4997-5002, 1998.

20. Meijerink, et al., Blood. 91: 2991-7, 1998.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A recombinant adenoviral vector encoding an pro-apoptotic bax gene.

2. A pharmaceutical composition, comprising the adenoviral vector of claim 1 and a pharmaceutically acceptable carrier.

3. A method of treating an individual having a pathophysiological state, comprising the step of administering to said individual a pharmacologically effective dose of the composition of claim 2.

4. The method of claim 3, wherein said pathophysiological state is a neoplastic disease.

5. The method of claim 4, wherein said neoplastic disease is selected from the group consisting of breast cancer, colon cancer, ovarian cancer, glioma, osteosarcoma and haemopoietic cancers.

6. The method of claim 4, wherein said composition is administered in a dose of from about 0.1 mg/kg to about 100 mg/kg.

7. A method of treating an individual having ovarian cancer, comprising the step of administering to said individual a pharmacologically effective dose of a pharmaceutical composition, comprising a recombinant adenoviral vector encoding an pro-apoptotic bax gene and a pharmaceutically acceptable carrier.

8. The method of claim 7, wherein said composition is administered in a dose of from about 0.1 mg/kg to about 100 mg/kg.

9. A method of sensitizing tumor cells to chemotherapy and/or radiotherapy in an individual, comprising the step of administering to said individual a pharmacologically effective dose of a pharmaceutical composition, comprising a recombinant adenoviral vector encoding an pro-apoptotic bax gene and a pharmaceutically acceptable carrier.

10. The method of claim 9, wherein said composition is administered in a dose of from about 0.1 mg/kg to about 100 mg/kg.
